(19) United States
(12) Patent Application Publication
(10) Pub. No.: US 2008/0097618 A1
(43) Pub. Date: Apr. 24, 2008

(54) Deposition of calcium-phosphate (CaP) and calcium-phosphate with bone morphogenic protein (CaP+BMP) coatings on metallic and polymeric surfaces

(76) Inventors: Kevin Charles Baker, Royal Oak, MI (US); Jaroslaw Wieslaw Drelich, Houghton, MI (US)

Correspondence Address: VAN OPHEM & VANOPHEM, PC;REMY J VANOPHEM, PC, 51543 VAN DYKE, SHELBY TOWNSHIP, MI, 48316-4447, US

(21) Appl. No.: 11/731,944

(22) Filed: Apr. 2, 2007

Related U.S. Application Data
(60) Provisional application No. 60/852,545, filed on Oct. 18, 2006.

(51) Int. Cl. A61F 2/28

(52) U.S. Cl. 623/23.51; 427/2.26; 435/402

(57) ABSTRACT

The invention is a medical implantable device which is coated by the method according to the invention. The surface of the substrate used for the implantable device, in the raw condition, following a cleaning regime and physiochemical pretreatments, is coated using a biomimetic process in a supersaturated calcium phosphate solution (SCPS) to obtain the desired coating coverage and morphology maintaining a ratio of calcium to phosphorus pH, as well as solution temperature plays a major role in yielding precipitation of the proper phase of CaP so that composition, morphologies, crystal structures, and solubility characteristics are optimal for the deposition process. The biomimetic coating adds the attribute of osteoconductivity to the implant device. To maximize bone growth, the implant must also induce bone growth, or possess the attribute of osteoinductivity. This attribute is acquired by the use of therapeutic agents, i.e. bone morphogenic proteins (BMP), growth factors, stem cells, etc. The preparation of the SCPS solution is slightly altered so that during the immersion of the implant in the SCPS, the therapeutic agents are co-precipitated and bonded with the CaP directly on the underlying surface of the implant device. A final dipping process into a BMP solution provides an initial burst of cellular activity. For delivering stem and/or progenitor cell, after drying the dipped solution of BMP, the cells are cultured on the surface of the implant.

### CROSS REFERENCES TO RELATED APPLICATIONS

The application claims the benefit of Unites States Provisional Application Ser. No. 60/852,545, filed on Oct. 18, 2006.

### FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The present invention relates in general to orthopaedic implant devices having surface coatings, and in particular to a method of making a prosthetic bone implant having a calcium phosphate coating which acts as a delivery vehicle for therapeutic agents and/or acts as a scaffold for the growth of soft tissue

2. Description of the Prior Art

Metallic materials have been used in the fabrication of orthopaedic devices since the middle of the 20^{th} century. The rigidity of these early constructs offered great potential in correcting deformities resulting from trauma, or congenital disorders. With advances in the aerospace industry, new alloys were introduced that quickly found applications in fracture fixation, joint reconstruction and spinal fusion. CoCrMo alloys are used commonly as hard bearing surfaces in knee and hip arthroplasty. Large-scale spinal deformity correction procedures call for a rigid material, such as 316L stainless steels. Structural components used in spinal fusion, or joint arthroplasty procedures are often fabricated from commercially pure titanium, or titanium alloys.

Polymeric materials were introduced to the field of orthopaedics soon after metallic materials. With improved elastic behavior and friction properties that approximated tissue from synovial joints, polymeric biomaterials quickly gained widespread acceptance by surgeons. Currently, the most common application for polymers in orthopaedics is in joint arthroplasty. Polymers such as ultra high molecular weight polyethylene (UHMWPE) act as bearing surfaces in knee, hip, shoulder, ankle and spinal arthroplasty and articulate against CoCrMo alloys, alumina ceramics, zirconia ceramics, or against other UHMWPE bearings. Poly-ether ether ketone (PEEK) has recently gained popularity for use in spinal fusion applications due to the similarity in elastic moduli between the polymer and bone. This similarity in elastic properties reduces the incidence of a phenomenon referred to in prior art as stress shielding. During stress shielding, the material with the greater elastic modulus bears the greater percentage of the load and leads to disuse atrophy of the bone.

Bioabsorbable polymers such as poly(L-lactic acid) (PLLA) have recently been introduced to the field of orthopaedics as a novel method to reduce the occurrence of stress shielding. In addition to a lower inherent elastic modulus, bioabsorbable polymers degrade in vivo in a predictable manner, which facilitates load sharing between the device and the bone. As the polymer degrades via hydrolysis and is excreted via natural physiological functions, a greater percentage of the biomechanical load is shifted to the bone guaranteeing successful remodeling. These polymers have been used extensively in plastic reconstruction of cranial defects and as bone graft substitutes for orthopaedic trauma and spinal fusion applications.

While many of the biomaterials used in the orthopaedic industry today have been chosen mostly based on their mechanical properties, these systems must also be biocompatible in their bulk form. Immune and inflammatory responses to non-compatible materials can have devastating consequences including infection, the need for secondary surgeries and even death. Fortunately, extensive biological testing of these materials in their bulk form has revealed that they are, for the most part, biologically inert. While this lack of interaction is desired from an immunological perspective, it is this same lack of bioactivity that requires additional fixation of the devices to the hard tissue of the patient.

The fixation of metallic, polymeric and ceramic materials must be achieved through physical machining, controlled oxidation, or by the addition of bone cement.

Many prior art devices achieve fixation by the addition of nails, or screws, which mechanically attach the device to the hard tissue. Acetabular components used in total hip arthroplasty, as shown in FIGS. 1a-1d, are held in the correct anatomical position by screws. The screws go through the holes at the back of the acetabular cup and into the hard tissue of the patient. The presence of these screws may predispose the polyethylene to damage, in a process referred to as backside wear. While the screws do provide adequate mechanical fixation in many instances, the generation of polyethylene wear particles does increase the likelihood of loosening of these screws (osteolysis).

Increasing the surface roughness of implants increases the surface area of the interface between the implant and surrounding hard tissue. The greater the contact area, the more of the implant that is integrated with bone, which increases the stabilization of implants in situ. Other researchers have also noted that there exists a preferential range of surface roughness on the nano-scale that will actually promote the differentiation of osteoblast cells.¹ Promotion of differentiation of osteoblasts enhances the osseointegration process and may lead to a long-term bony-union between the host and implant. Machining relies solely on the manipulation of implant surface topography. However, biological systems also rely on chemical information as a mode of interpretation of its surrounding.

Initial research as taught by a study by the Work Committee for Implants of the German Society of Material Testing published in 1987, suggested that contact surface roughness was the solution to adequate adhesion between bone and implant since smooth contact surfaces of titanium implants did not provide adequate interfaces that would resist tension forces. The prevailing opinion was that contact surface roughness of more than 20 μm was required. Subsequently, Steinemann, in U.S. Pat. No. 5,456,723 taught any implant of titanium or of another similar material is to have a contact surface roughness of 2 μm or less to yield a good bond between bone and implant. Such roughness was taught to be readily produced by subjecting the contact surface to pickling in a reducing acid. Later work performed by Kokubo et al., has dealt with the oxidation of titanium alloys to yield specific phases of TiO₂, mainly anatase, that have been shown to promote positive biological interaction with host environments through changes in both surface topography and chemistry.²⁻⁶ Heat treatments in various atmospheres were the first method employed to oxidize the titanium alloy implant materials.⁴⁻⁶ Alkali and acid treatments were also successfully implemented by a number of researchers.⁴⁻⁶ The main focus in both research and industry right now is the effective use of anodic oxidation, or anodization.^{2,3} Anodized Ti6Al4V substrates are the current industry standard for pedicle screws for instance.

Oxidation of polymeric materials used for orthopedic applications has not gained widespread acceptance as a method by which implant stabilization can be increased. Since the discovery that sterilization techniques such as ethylene-oxide vapor processing actually reduce the fracture toughness of polyethylene components, much has been done to prevent oxidation of these materials, rather than investigate possible benefits. Alkali and acid pretreatments, ultra-violet irradiation and glow discharge processing are all methods currently employed in various non-medical industries to improve the wettability of print-accepting surfaces. It should follow that improving the surface characteristics of these materials would also improve their in vivo performance.

Acrylic based cements, most notably poly-methyl-methacrylate (PMMA), have gained widespread use for immediate fixation of implants. PMMA cements are often used to fixate femoral stems, acetabular cups, humeral stems and glenoid components. Since the curing process is rapid, the cements are often mixed right before implantation of the device. The mixing is usually performed by the surgeon or a surgical technician so there may be variation in the degree of mixing of the cement components, or in the application of the cement to the implant itself. These variations have been blamed for numerous implant failures.⁷⁻⁹

Regardless of the mixing, or application of the cement, the curing process is largely exothermic. Heat released during the reaction is absorbed by the host tissue and implant material. Recently, it has been shown that the use of PMMA bone cements has lead to death of bone cells in the vicinity of the implantation site.⁷⁻⁹ The high temperatures also cause a retreat of mineralized tissue away from the heat source, which leads to implant loosening. Implant loosening is a major source of pain for patients receiving arthroplasty due to the increased generation of wear debris and disrupted biomechanics of the joint.⁷⁻⁹

Bioactive calcium-phosphate (CaP) coatings have been employed in several orthopaedic applications to provide an environment conducive to bone growth at the surface of implants.¹⁰⁻¹⁷ Plasma sprayed hydroxyapatite (HA) is the most common form of bioactive coating used to enhance hard tissue integration with orthopedic implants. Since the plasma spray deposition method is a line of sight process, coatings produced on implants with complex geometries (screws, interbody fusion cages, etc.) are often non-uniform in terms of substrate coverage and coating thickness.¹⁰ The plasma spray deposition process also entails the use of high temperatures which can lead to heterogeneous coating properties and increased crystal sizes. Increased crystal sizes effectively reduce nano-scale surface roughness, which has been shown to negatively impact the state of differentiation of osteoblast cells.¹ To solve these problems, research has focused on biomimetic processes, which have simple chemical immersion techniques as their basis. During the biomimetic deposition process, a substrate is immersed in a solution saturated or supersaturated with calcium and phosphate ions. The substrates remain immersed in the solution for a specified amount of time. A method by which CaP films can be biomimetically deposited on metallic and polymeric materials is described in detail, elsewhere.¹⁰

Leitao et al., in U.S. Pat. Nos. 6,143,948, 6,136,369 and 6,344,061 focused on a biomimetic process, which has simple chemical immersion techniques as their basis. Leitao et al. relied very strongly on acquiring a specific suggested surface roughness on the substrate and considers surface roughness of the substrate a critical factor in achieving a suitable implant in order to give rise to the formation of a composite coating when placed in certain solutions. Leitao et al. disclosed that the most suitable roughness for the substrate surface is a direct function of the nature of the material of the substrate. For implants made of titanium, the average peak distance, i.e. the average spacing between protrusions on the surface (Rₐ value) as determined by means of a scanning electron microscope (SEM), can be from 10 to 200 μm, for polymeric material, the preferred peak distance is from 20 to 500 μm, whereas for stainless steel the peak distance is advantageously between 50 and 1000 μm. Leitao et al. further discloses that the depth of the surface roughness of the implant is less critical than the peak distance. However, a minimum depth is desirable, in particular a peak height of at least 20 μm, up to about 2000 μm. The preferred average depth is of the same order of magnitude as the average peak distance, and is in particular from 50 μm to 1000 μm.

The substrate with the desired physical topography is then coated in vitro with a layer of calcium phosphate and one or more biologically active agents by a very time consuming process. The bulk of the time expenditure is principally due to the step of immersing in a simulated body fluid at 37° C. for 14 days in separate polyethylene containers. Leitao also requires a fluid change every 48 hours, presumably to overcome the detriment to further nucleation and growth due to ion consumption.

Leitao et al. further discloses that in the formation of the implantable device disclosed with specific contact surface roughness to facilitate in vitro formation of a solution mediated coatings, biologically active substances can be co-precipitated such that the device acts as a delivery system for the efficient application of therapeutic agents.

There is therefore a strong need to discover materials for coating implantable medical implants that are industrially viable by reducing production time and costs. These implants are entirely biocompatible and thus do not cause any adverse effects on the tissue. Ideally this substrate acts as a delivery system for biologically active substances and is degradable predictably in vivo by cellular activity and hydrolytic reactions. By-products of degradation are innocuous and removed via normal physiological processes. Since bio-absorbable polymers inherently have lower moduli than metallic materials, such as titanium or stainless steel alloys, stress-shielding will be reduced or eliminated since degradation of the polymer also predicates load-sharing between the device and the host system over time. Furthermore, the ideal coating material will be able to deliver one or more pharmaceutically active agents to a targeted site. The drug release properties can be used to shield the implant device, to some extent, from both cellular activity and the aqueous environment. Increased rates of bone growth induced by bioactive coatings may also reduce the difference between device degradation time and hard tissue remodeling time.

In order to gain widespread industrial implementation, processes of deposition being similar, significant improvements must be made in substrate pretreatments to enhance coating coverage and adhesion so as to increase the industrial viability of the process by reducing production time and associated costs.

### BRIEF SUMMARY OF THE INVENTION

The invention is a medical implantable device which is coated by the method according to the invention. The surface of the substrate used for the implantable device, in the raw condition, following a cleaning regime and physiochemical pretreatments, is coated using a biomimetic process in a supersaturated calcium phosphate solution (SCPS) to obtain the desired coating coverage and morphology maintaining a ratio of calcium to phosphorus, pH of solution, as well as solution temperature plays a major role in yielding precipitation of the proper phase of CaP so that composition, morphologies, crystal structures, and solubility characteristics are optimal for the deposition process. The biomimetic coating adds the attribute of osteoconductivity to the implant device. To maximize bone growth, the implant must also induce bone growth, or possess the attribute of osteoinductivity. This attribute is acquired by the use of therapeutic agents, i.e. bone morphogenic proteins (BMP), growth factors, stem cells, etc. The preparation of the SCPS solution is slightly altered so that during the immersion of the implant in the SCPS, the therapeutic agents are co-precipitated and bonded with the CaP directly on the underlying surface of the implant device. A final dipping process into an aqueous solution containing pre-selected therapeutic agent provides an initial burst of cellular activity. For delivering stem and/or progenitor cell, after drying the dipped solution therapeutic agent, the cells are cultured on the surface of the implant.

The effect of UV irradiation and glow discharge processing of the polyethylene (PE) substrates on deposition of calcium phosphate (CaP) films from supersaturated aqueous calcium phosphate solutions was investigated. CaP coatings deposited on the PE substrates consist of elongated clusters of spherical particles and 100% of the free surface area of nearly all of the substrates was covered with a porous CaP film after a three day immersion. Nano-scratch tests determined that PE-CaP adhesion was most improved when PE substrates were subjected to 50 W glow discharge treatments. As determined through contact angle measurements, the glow discharge treated PE samples had the highest electron donor parameter of surface energy characteristic suggesting that enhancing the electron donor parameter of PE leads to improved adhesion with the biomimetic CaP coating.

It is an object of the invention to provide a medical implant device that significantly reduces a patient's healing time.

It is a further object of the invention to provide a medical implant device that combines the attributes of osteoconductivity with the attributes of osteoinductivity to not only act as a scaffold for bone growth on the implant but also to induce bone growth at the fusion site.

It is yet a further object of the invention to provide a medical implant device that utilizes a biomimetic coating process directly deposited onto the substrate material in its raw state, without time consuming, or capital intensive surface finish processes.

It is yet a further object of the invention to provide a medical implant device by way of a biomimetic deposition process of CaP coatings from supersaturated calcium phosphate solutions.

It is yet a further object of the invention to provide a medical implant device to provide a biomimetic coating that enhances osseointegration of implantable devices.

It is yet a further object of the invention to provide a biomimetic coating that enhances bone tissue growth and arthrodesis.

It is still a further object of the invention to provide a composite biomimetic coating that delivers growth factors, proteins, antibiotics, and stem cell (marrow stem cells, osteoprogenitor cells, progenitor cells, etc.).

It is a further object of the invention to provide a medical implant device wherein physiochemical manipulation of the surface functionality of metallic, polymeric, ceramic, or organic-based substrate materials enhances the amount and density of potential CaP nucleation sites, without a prominent effect on surface topography.

It is yet a further object of the invention to provide a method of making an implant device which dramatically reduces costs associated with the manufacturing of implantable devices.

It is yet a further object of the invention to provide a polymeric medical implant device wherein the substrate, prior to the biomimetic coating process, is exposed to alkali pretreatments and glow-discharge processing to increase the deposition rate and substrate-coating adhesion by reducing apatite formation induction time and thereby enhances substrate adhesion on polymers.

These objects and other features, aspects, and advantages of this invention will be more apparent after a reading of the following detailed description, appended claims, and accompanying drawings.

### BRIEF DESCRIPTION OF THE DRAWINGS

FIGS. 1a-1d are photographs of a retrieved prior art total hip arthroplasty implant showing the total assembly, FIG. 1a, polyethylene liner in a metal acetabular cup, FIG. 1b, a metal femoral head articulating against a polyethylene liner in a metal acetabular cup, FIG. 1c, and a porous titanium bead coating on the metal acetabular cup, FIG. 1d;

FIGS. 2a and 2b are photomicrographs of representative images of topography of 2×2 μm² section of a glass slide sample, (FIG. 2a) and a 20×20 μm² polystyrene tensile bar section sample, (FIG. 2b) respectively using an atomic force microscope;

FIGS. 3a-3f are photomicrographs of scanning electron microscope (FE-SEM) images of CaP films possessing different surface functionalities, COOH (FIG. 3a); OH (FIG. 3b); CH₃ (FIG. 3c); COOH+OH (FIG. 3d); COOH+CH₃ (FIG. 3e); and OH+CH₃ (FIG. 3f) biomimetically deposited on self-assembled monolayers of alkanethiols (SAMs), under Type I immersion technique;

FIGS. 4a and 4b are photomicrographs of a FE-SEM micrograph of a CaP coating on COOH-terminated SAMs highlighting the detail of coating morphology using a Type I immersion technique;

FIG. 5 is a photomicrograph of a FE-SEM micrograph of CaP particles separated from supersaturated calcium-phosphate solution (SCPS) in a glass micro-fiber filter;

FIGS. 6a-6g are photomicrographs of a FE-SEM micrograph of CaP coatings biomimetically deposited on SAMs possessing different surface functionalities, COOH (FIG. 6a); OH (FIG. 6b); CH₃ (FIG. 6c); OH+COOH (FIG. 6d); CH₃+COOH (FIG. 6e); OH+CH₃ (FIG. 6f); and OH+COOH+CH₃ (FIG. 6g); terminated by Type IIB SCPS immersion techniques;

FIGS. 7a-7d are photomicrographs of a FE-SEM at 30,000× magnification of images exhibiting different surface functionalities COOH(2):OH(1) (FIG. 7a); COOH(1):OH(2) (FIG. 7b); COOH(1):OH(4) (FIG. 7c); and COOH(1):OH(10) (FIG. 7d) of CaP coatings prepared on heterogeneous SAM under Type IIA immersion techniques;

FIGS. 8a and 8b are photomicrographs of FE-SEM images of a CaP coating on COOH-terminated SAMs using Type IIB SCPS immersion technique;

FIG. 9 is a photomicrograph of a FE-SEM image of a prior art CaP coating deposited on Ti-6Al-4V alloy immersed in a supersaturated calcium phosphate solution;

FIGS. 10a and 10b are photomicrographs of an atomic force microscope (AFM) image of the topographic (FIG. 10a) and phase (FIG. 10b) image of an untreated polyethylene surface irradiation;

FIGS. 11a and 11b are photomicrographs of an AFM phase images of a polyethylene (PE) sample treated with UV irradiation for 60 minutes (FIG. 11a) and Glow Discharge processing at (50 W) for 30 seconds (FIG. 11b);

FIGS. 12a-12c are photomicrographs at 30,000× magnification of SEM images of CaP particle morphologies on polyethylene (PE) substrates having undergone no pretreatment (FIG. 12a) UV irradiation treatment for 10 minutes (FIG. 12b) and a 50 W Glow Discharge treatment for 10 seconds (FIG. 12c) the scale bar shown is 1 μm;

FIGS. 13a-13c are photomicrographs at 15,000× magnification of SEM images of CaP coatings on polyethylene substrates having undergone no pretreatments (FIG. 13a) UV irradiation for 10 minutes (FIG. 13b) and a 50 W Glow Discharge treatment for 10 seconds (FIG. 13c). Scale bar shown is 2 μm;

FIG. 14 is a graph of an x-ray diffraction scan of a CaP coated polyethylene substrate;

FIG. 15 is a graph of an infrared spectrum of a CaP coated polyethylene substrate; and

FIG. 16 is a photomicrograph of an SEM image of a nano-scratch test region for the CaP coated polyethylene substra. The points of initial load application and coating delamination are illustrated by circular dots.

### DETAILED DESCRIPTION OF THE PREFERRED EMBODIMENT

The present invention overcomes various drawbacks in the prior art by proposing an improved method and apparatus of depositing calcium phosphate coatings for use in orthopaedic applications. These applications include, but are not limited to:
1. Surface coatings to enhance osseointegration of implantable devices;
2. Surface coatings to enhance and stimulate bone growth and arthrodesis; and
3. Composite coatings to deliver growth factors, proteins, antibiotics, stem cells (marrow stromal cells, osteoprogenitor cells, progenitor cells, etc.).

The current process overcomes drawbacks in prior art by focusing on surface chemical functionality as the primary factor in improving the deposition and substrate-coating adhesion characteristics of calcium phosphate-based films. Careful physiochemical manipulation of the surface functionality of metallic, polymeric, ceramic, or organic-based materials enhances the amount and density of potential CaP nucleation sites, without a prominent effect on surface topography.

Since surface chemistry, not topography is the primary factor effecting deposition and adhesion of CaP films, substrate materials can be used in their “as-machined” or raw state, without time-consuming, or capital intensive surface finish processes. This has the potential to dramatically reduce costs associated with the manufacturing of implantable medical devices. Compared to prior art, such as that put forth by Leitao et al., the current process decreases the number and duration of substrate pretreatments, which is another potential source of cost reduction. The current process also relies on the immersion of a substrate in a supersaturated calcium phosphate solution (SCPS) at ambient temperatures, in place of the more chemically complex simulated body fluid (SBF) at physiological temperatures (37° C.) used in prior art. Reducing the chemical complexity of the immersion solution eliminates the possibility of side-reactions that may occur when the process is scaled-up to industrial scale and places emphasis on the interaction between the substrate and the ions in solution. Ambient immersion temperatures eliminate the additional energy expenditures required to elevate and maintain solution temperatures at, or above physiological conditions.

Coating-substrate adhesion is a major problem facing the use of biomimetic CaP films with polymeric materials. Chemical etching, flame treatment, ultraviolet (UV) irradiation, corona treatment, and plasma treating are all methods currently employed in various non-medical industries to improve the adhesion of polymers with other materials. Over the years, similar treatments of polymers have been tested in laboratories for biomedical applications. Many reports suggest that surface polar groups are created during these processing steps and act as nucleation sites for the formation of CaP structures and increase the rate of CaP deposition.^{11-12,18-19} For example, exposing the substrate to UV radiation prior to immersion in a simulated body fluid solution has been shown to enhance substrate-apatite adhesion properties.¹⁹ The exposure of a substrate to UV radiation increased the density of polar groups at the substrate surface, which interact favorably with CaP nuclei.¹⁹ Alkali pretreatments and glow-discharge (GD) processing of substrates prior to biomimetic formation of CaP coatings also increased deposition rate and substrate-coating adhesion through a similar mechanism.¹¹⁻¹² It is interesting to note, however, that GD processing was the only method that reduced apatite formation induction time and enhanced substrate adhesion on polymers such as polyethylene terephthalate (PET), poly(methyl methacrylate) (PMMA), polyamide 6 (PA6), polyethersulfone (PESF), polyethylene (PE) and polytetrafluoroethylene (PTFE), while other treatment methods were only effective for certain polymers.^{11-12,19}

It is therefore, rational to assume that the surface chemistry plays a key role in the formation and growth of biomimetic CaP films. To understand the effect of surface chemistry on nucleation, growth, and adhesion of CaP films, self-assembled monolayers (SAMs) of alkanethiols possessing differing surface functionalities were prepared on gold-coated glass and polystyrene substrates. The monolayers served as model organic substrates in the biomimetic deposition of CaP coatings from supersaturated calcium phosphate solutions. Methyl (CH₃) terminated SAMs were meant to serve as a plausible model of a saturated hydrocarbon surface, such as polyethylene (common biomaterial). SAMs with oxygen-based functionality, either homogeneous (COOH, OH) or heterogeneous (OH+COOH, CH₃+COOH, OH+CH₃, OH+COOH+CH₃), were selected to simulate organic polymer surfaces that had been modified by a generic oxidation pretreatment. Tanahashi and Matsuda also used SAMs in studying the formation of apatite structures from a simulated body fluid.²⁰ They found that the growth rate of apatite coatings on SAMs with PO₄H₂ and COOH functional groups was substantially higher than for SAMs with CONH₂, OH, and NH₂, groups. There was practically no growing of the apatite structures on SAMs with methyl as the end functionality. In other studies on the formation of HA on Langmuir-Blodgett monolayers, Sato et al. reached a similar conclusion.²¹ The authors found nucleation and growth of HA on monolayers with carboxyl groups and practically no nucleation on the monolayers with amino groups.

In contrast to the above previous prior art endeavors, we investigate the influence of chemical functionality on the CaP coating morphology and substrate coverage when the biomimetic deposition process is carried out using supersaturated calcium phosphate solutions (SCPS) instead of simulated body fluids. Our previous research showed that the deposition rate for CaP coatings is accelerated from supersaturated solutions.¹⁰ To accomplish the stated objectives as set forth heretofore, it was necessary to first understand the influence of substrate surface chemistry and immersion techniques on the formation and morphology of CaP coatings in general. With this understanding, a specific method by which polyethylene components can bond directly to bone would be formulated.

### Experimental Procedure

### Substrates

High impact polystyrene (PS) (available from Dow Chemical) tensile bars were sectioned into 17×17 mm squares using a silicon carbide cutting wheel and smoothed using a surgical scalpel. Glass slides (GS) (obtained from Eerie Scientific) were cut into 37×25 mm rectangles using a diamond scalpel. Both PS and GS samples were subjected to ultrasonic cleaning in a diluted surfactant solution (Micro 90, manufactured by International Products Inc.) for five minutes. The samples were then rinsed with de-ionized water and ultrasonically cleaned in methanol (histological grade, obtained from Fisher Scientific) for an additional five minutes. After ultrasonic cleaning, the GS samples were rinsed once more with methanol and acetone (99.9%, Fisher Scientific) placed in a covered Pyrex dish and allowed to dry in an L-C oven for at least 1 hour at 100° C. The PS samples were rinsed with methanol (histological grade, Fisher Scientific) for 30 seconds following the ultrasonic cleaning and dried in the L-C oven at 70° C. for 15-20 minutes.

Both sides of the GS and PS samples were sputter-coated with a 2.5 nm thick gold film (Hummer 6.2, manufactured by Anatech LTD.). The average pressure of the vacuum was 0.075 Torr with a plasma discharge current of 0.015 amps during deposition.

### Preparation of Self-Assembled Monolayers

Formation of SAMs on the sputtered gold surfaces took place from 1-10 mM solutions of alkanethiols in histological grade methanol at 22-25° C. Hexadecanethiol (HS(CH₂)₁₅CH₃, MW=258.51 g/mol, 95.0%, available from Fluka Chemical), 16-mercaptohexadecanoic acid (HS(CH₂)₁₅COOH, MW=288.50 g/mol, 90.0%, (available from Aldrich) and 11-mercapto-1-undecanol (HS(CH₂)₁₁OH, MW=204.24 g/mol, 97.0%, (available from Aldrich) were used without further purification.

SAMs with CH₃, OH, or COOH end functionality were prepared on gold-coated GS and PS substrates by immersion in 50 mL of respective thiol solutions in covered beakers for approximately 36 hours. Heterogeneous SAMs (CH₃+COOH, OH+COOH, OH+CH₃, or OH+COOH+CH₃) were also prepared according to the same protocol using a mixture (1:1, 2:1, 1:2, 1:4 or 1:10) of respective solutions. Substrates with SAMs were rinsed with methanol to remove organic debris and physically adsorbed thiols. They were then placed in covered Pyrex dishes and dried in an L-C oven at 100° C. for at least 15 minutes. The substrates were stored in clean, covered Pyrex dishes for a maximum of three hours before they were immersed into supersaturated CaP solutions.

### Characterization of Self-Assembled Monolayers

The self-assembled monolayers were characterized by measurements of advancing and receding (static) water contact angles using the sessile drop technique.²² A syringe equipped with a 0.5 mm Luer-tipped needle was filled with deionized water (18MΩ). A small drop was placed on the solid surface of interest and adding more water from the syringe increased its volume. When the size of the drop was increased, the three-phase contact line advanced over the dry solid surface creating conditions for the measurement of advancing contact angle. Measurements of advancing contact angles were carried out 10-15 seconds after enlargement of the drop size to allow the three phase contact line to stabilize its shape. Then, the needle tip was carefully lowered into the sessile drop and water was removed to reinforce the reduction in the length of the three-phase contact line and create conditions for receding contact angle measurements. The receding contact angles were measured 10-15 seconds after change in the drop volume was made. A Kruss-G10 goniometer with drop shape analysis software was used for measuring seven advancing and seven receding contact angles. Average values and their standard deviations are shown in Table I below.

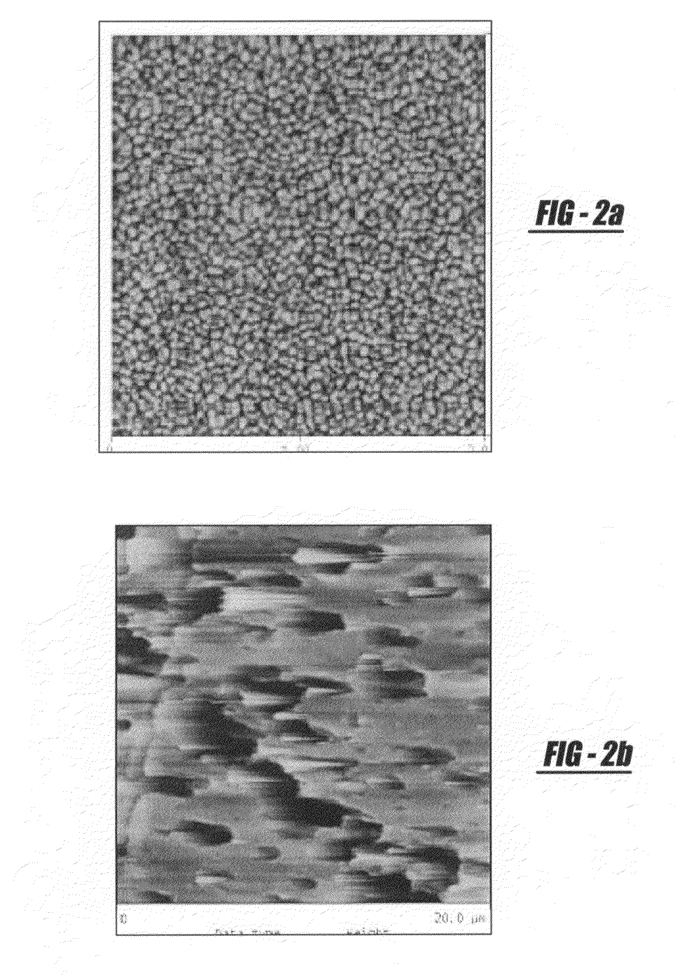

Surfaces of selected substrates were imaged with the Dimension 3000 atomic force microscope (AFM, available from Digital Instruments). A Si₃N₄ cantilever tip (300 kHz, 40N/m) in intermittent contact mode was used. Scan sizes from 2×2 μm to 20×20 μm² were used for characterization of surface roughness and asperity geometries as illustrated in FIG. 2.

### Formation of Calcium Phosphate Coating

A supersaturated calcium-phosphate solution (SCPS) was prepared by combining 5.6 mM CaCl₂.2H₂O (MW=147.02 g/mol) (available from Fisher Scientific) and 3.34 mM NaH₂PO₄.H₂O (MW=137.99 g/mol, 99.4%) (available from Fisher Scientific) in 1:1 volumetric ratio. The resting pH of the solution was approximately 3.2 and a tris-buffer (tris(hydroxymethyl)-aminomethane, C₄H₁₁NO₃, MW=121.14 g/mol, 99+%, (available from Aldrich) was used to elevate and maintain the solution pH to approximately 7.35. Solution pH was monitored during preparation (pH-Meter, available from Metrohm Inc.).

Chemical immersion in the SCPS took place in one of three ways listed below.

Type I Immersion. This is a combined immersion technique, in which all samples were immersed in an 800 mL beaker containing 700 mL of SCPS. Beakers were covered during immersion by a sheet of paper to reduce dust and foreign objects in the solution. The samples were positioned by placing them in plastic microscope slide holders. Although orientation and position were held constant during the experiment, the samples were not perfectly vertical throughout immersion and faced the bottom of the beaker at an angle of 65-70 degrees. Using this technique sample surfaces were between 5 and 8 mm from each other. Surfaces facing downwards (not predisposed to sedimentation) were analyzed during characterization.

Type II Immersion. In the first set of experiments using the Type I immersion technique, it was found that the orientation of the sample has a profound effect on formation of CaP deposits and coatings. In order to avoid the accumulation of CaP precipitates on samples from supersaturated solutions, a vertical orientation was maintained throughout immersion. In this technique two different approaches were used when individual samples, or a set of a few samples were immersed into the SCPS solution.

Type IIA. Samples were immersed vertically in individual 50 mL beakers containing approximately 50 mL of SCPS. The beakers were covered with a thin polyolefin film throughout immersion. The SCPS was poured from a single batch that underwent constant stirring between dispersions into each beaker to ensure solution homogeneity. Vertical immersion was achieved either by implementing metal hangers attached to both the sample and to the parafilm covering the beaker, or by painted metal clips that held the samples vertical in solution. Distance of the samples from the sides of the glass beakers, which were being used as reaction cells, was held nearly constant throughout experimentation.

Type IIB. This is an improved Type IIA immersion technique that eliminates difficulties associated with hanging samples at perfect vertical orientation. Painted metal clips, that underwent a cleaning regime similar to that used for the actual samples, were attached to the bottom of PS samples with minimal obstruction of surface area. The clips were shaped in a manner such that they allowed the samples to be perfectly vertical while in solution. Immersion took place in a 100×50 mm Pyrex dish containing approx. 250 mL of SCPS with even spacing between samples that exceeded 3 cm. The dish was covered with a glass lens during experimentation. From 4 to 8 samples could be placed in the same solution during experimentation, securing the same deposition conditions for each sample.

In each experiment, Type I and II, samples remained immersed in the SCPS for three days with daily solution refreshments. During solution refreshments, the old solution was carefully pipetted out of the beaker(s). New SCPS was carefully poured back in making sure not to disturb the samples. After three days of immersion, the samples were removed and gently washed with de-ionized water, placed in a covered Pyrex dish and dried in an L-C oven at 75° C. for 15 minutes.

### Characterization of Calcium-Phosphate Coatings

Morphology and coverage of calcium phosphate coatings were examined under scanning electron microscopy, using a JEOL6400 SEM instrument. Approximately 12 mm wide sections were cut from the GS samples by a diamond scalpel. The size of the PS samples did not dictate that they be sectioned for observation. Samples were then mounted to aluminum pucks and sputter-coated with 2.5 nm of Au—Pd film to enhance resolution and reduce charging effects during SEM. SEM imaging was performed at an accelerating voltage of 20 kV and a working distance of 8 mm. Images were gathered via the digital image acquisition system in place with 1000 pixels per line and a dwell time of 0.1 msec for optimal image quality.

Field-emission scanning electron microscopy (FE-SEM, Hitachi S-4700, available from Hitachi Inc.) was employed to more closely examine structures of deposited CaP films. An accelerating voltage of 5 kV and working distance of either 5.6 or 10.5 mm was used to resolve nanometer-scale features within the films.

The SEM and FE-SEM images were analyzed using Scion Image 4.02 (manufactured by Scion Corporation) to gather information about the size of CaP particles/structures in the coatings.

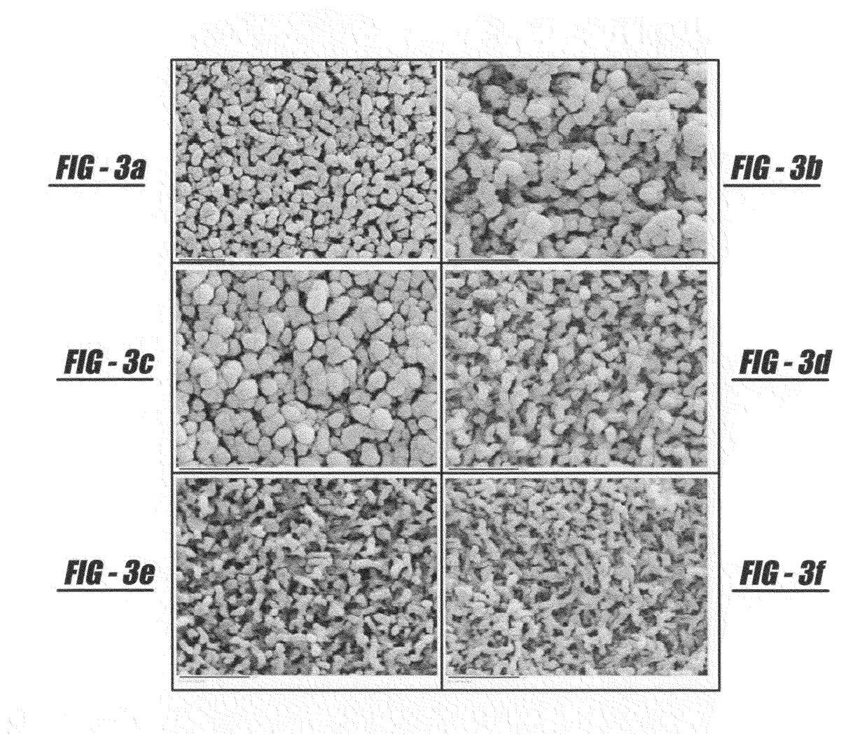

Energy dispersive x-ray spectrometer (EDS) installed on the JEOL6400 SEM instrument was used to gather information about the calcium to phosphorus ratio of the deposited coatings. Carbon sputtering was used instead of Au—Pd sputtering for EDS analysis as the M₅-peak for gold (2.2057 keV) interfered with the K_{α}-peak of phosphorus (2.1455 keV). EDS analysis was performed at 20 kV of accelerating voltage with a working distance of 39 mm and a magnification of 100,000×. A live time of 100 seconds and dead time setting of approximately 22% was used for all compositional characterization with the EDS. Measurements were made in five separate locations on each sample using the same settings and average value and standard deviation are shown in FIG. 3. Information about the calcium to phosphorus ratio was obtained by the quantitative microanalysis tool inherent within the spectrum gathering software (Spirit Software).

A 31×41 transparent grid was super-imposed over SEM micrographs representative of the morphology of CaP films produced by Type I and Type IIA immersion techniques on the monolayers. “Void fraction” in the porous coating was determined as the ratio of grid points that fell on pores and the total number of grid points. These measurements are presented as percentage of surface area of the images.

### Precipitates from Supersaturated Calcium Phosphate Solution

CaP particles were separated from one of the supersaturated solutions for evaluation of their size and morphology. An SCPS solution was prepared and poured through a glass micro-fiber filter (0.5 μm pore size, Whatman) under suction. The filter was then washed with acetone to remove water and placed in an L-C oven at 75° C. for 48 hours prior to observation by FE-SEM. Imaging of particles was carried out with FE-SEM at accelerating voltage of 5 kV and working distance of 5.6 and 10.5 mm. The particle size was determined from collected images using Scion Image 4.02 software.

### Results and Discussion

### Substrate Surface Characteristics

FIGS. 2a and 2b show representative AFM images of GS and PS samples at different resolution. Both types of samples possessed nano-scale roughness such as shown in the AFM image in FIG. 2a for the GS sample. However, the nano-roughness was not analyzed in detail for PS samples due to the dominance of microscopic and sub-microscopic roughness for this type of sample as shown in the AFM image of FIG. 2b.

Grainy texture of the GS sample surface shown in FIG. 2a is characteristic of glass slides coated with gold films by a sputtering technique and appearance of grainy structure results from the formation of nano-sized gold islands. From digital image analysis it was determined that the average diameter of the islands of gold was 56±12.6 nm and this dimension is very similar to what we reported previously for substrates prepared according to the similar procedure.²³ Roughness values were also determined for the GS sample. The sample had a root-mean squared roughness of RMS=3.7 nm, a geometrical roughness Rₐ=3.0 nm, and a roughness parameter (representing the ratio between actual and projected surface area) r=1.14.

Microscopic irregularities and characteristic “pitting” sites with a size of a few micrometers and variation in height of up to about 1 μm, characteristic of extruded amorphous-crystalline polymers, are evident on the surface of the PS samples (FIG. 2b). Roughness values determined for the PS sample are R_{RMS}=100 nm, Rₐ=73 nm, and r=1.13. The value of r is similar to that determined for the GS sample suggesting that nanoroughness had a major impact on the roughness parameter.

Contact angles measured for water drops on SAMs prepared in this investigation are shown in Table 1. Receding contact angle measurements were difficult to obtain on carboxyl and hydroxyl-terminated SAMs, as these values were near zero. The small profile of the receding drops challenged the ability of the drop shape analysis software to discern between the liquid and solid surface. Contact angles near zero are reported as smaller than 5 degrees.

TABLE 1
Advancing (Adv.) and receding (Rec.) contact angles for water
drops on self-assembled monolayers. (NA = not available; GS = self-assembled
monolayers prepared on gold-coated glass slides; PS = self-assembled monolayers
prepared on gold-coated polystyrene samples; 1:1 etc. stands for the molar ratio
of relevant thiols in solution used in preparation of SAMs and does not stand for
the ratio of functionality formed on the substrate)

| | Water Contact Angle [deg] | | | |
|---|---|---|---|---|
| | GS Sample | | PS Sample | |
| Surface Functionality | Adv. | Rec. | Adv. | Rec. |
| COOH | 47.5 ± 0.4 | 14.5 ± 0.9 | 23.6 ± 1.7 | <5 |
| OH | 46.4 ± 0.4 | 26.2 ± 2.0 | 38.2 ± 1.6 | <5 |
| CH₃ | 111.4 ± 2.7 | 88.0 ± 2.0 | 95.7 ± 0.8 | 83.7 ± 2.6 |
| OH + COOH (1:1) | 39.8 ± 1.1 | NA | 36.7 ± 1.4 | <5 |
| OH + COOH (1:2) | NA | NA | 37.6 ± 1.5 | 15.9 ± 1.2 |
| OH + COOH (2:1) | NA | NA | 55.9 ± 2.3 | 23.6 ± 4.5 |
| OH + COOH (4:1) | NA | NA | 56.0 ± 1.3 | 24.0 ± 1.0 |
| OH + COOH (10:1) | NA | NA | 59.2 ± 2.7 | 19.0 ± 2.4 |
| CH₃ + COOH (1:1) | 98.9 ± 5.3 | 61.6 ± 2.6 | 84.2 ± 2.5 | 50.8 ± 4.1 |
| OH + CH₃ (1:1) | 94.8 ± 1.9 | 76.0 ± 2.0 | 113.1 ± 0.8 | 87.4 ± 3.5 |
| OH + COOH + CH₃ (1:1:1) | NA | NA | 78.2 ± 1.2 | <5 |

The hydrophobic character of CH₃-terminated SAM is revealed by high advancing water contact angles, approximately 111 and 96 degrees for GS and PS samples, respectively. Typical advancing contact angle values measured for such SAMs are in the range of 110-112 degrees.²² Contact angles several degrees smaller recorded for CH₃-terminated SAM formed on the PS sample suggests less dense packing of thiol molecules in the monolayer structure and/or submicroscopic imperfections of this particular sample. Receding water contact angles of 84-88 degrees were measured and they are also indicative of certain imperfections of the samples used in this investigation. Because both GS and PS samples covered with CH₃-terminated SAM had different roughness characteristics, but still showed similar receding contact angles, we expect that the samples had some degree of heterogeneity. In our previous research, we speculated that this heterogeneity might result from the intergranular area of the gold coating that is uncovered with thiols.²³

COOH and OH-terminated SAMs served as hydrophilic surfaces in this investigation. Similar advancing water contact angles were recorded for both polar SAMs, COOH-terminated SAMs being more hydrophilic as revealed by smaller contact angles, particularly when prepared on gold-coated PS substrates. A value close to zero degrees for advancing water contact angles was reported for freshly prepared COOH and OH-terminated SAMs,²⁴ whereas as high as 84 and 65 degrees were reported in our previous paper.²³ Aging of the SAMs and its sensitivity to air-born contaminants are expected to be the major reasons for such a broad variation in measured contact angles for SAMs with COOH and OH functional groups. These relatively high-energy surfaces have a tendency to reduce their surface energy by adsorbing organics from laboratory air.

The low standard deviation for most of the measured contact angles and reasonable contact angle hysteresis values (difference between advancing and receding contact angles) are also indicative of a relatively good quality samples prepared in this investigation.

Any of the heterogeneous SAMs containing the methyl functionality produced large water contact angles suggesting that the thiols with methyl groups dominated the molecular structure of SAMs. The SAMs with mixed OH and COOH functionality demonstrated contact angles that are larger than contact angles measured on homogeneous COOH or OH-terminated SAMs. This result is of no surprise as 16-mercaptohexadecanoic acid is longer by four methylene units than 11-mercapto-1-undecanol and surfaces composed of these two thiols have triple surface functionality, two polar OH and COOH, and nonpolar CH₂.²⁵

### Morphology of Calcium Phosphate Coatings

Deposition characteristics of CaP on SAMs differed greatly depending on the conditions of immersion. Type I immersion experiments yielded CaP films with a spherical morphology, while Type IIa and IIb immersion methods produced films with a plate-like structure. The variance in morphology is indicative of a difference in the underlying deposition mechanism between the two immersion techniques and is discussed hereinafter.

Type I Immersion Experiment. The SEM images of CaP coatings obtained according to Type I SCPS immersion are shown in FIGS. 3a-3f. The coatings appear to be a network of many individual particles attached to each other that produce a porous structure. The clustered structure of Type I immersion CaP films may indicate a deposition mechanism based on solution-formed nuclei and subsequent deposition of particles.²⁶

The particles on the homogeneous SAMs were quite spherical in nature and differed between functionalities in their densities and packing structures. CaP particles on heterogeneous functionality SAMs deviated from sphericity showing an elongated structure with the OH+CH₃ SAM having the largest aspect ratio. Type II immersion techniques failed to produce coatings of similar structures as discussed below.

CaP particle sizes were analyzed from the SEM images and found to be 230±21 nm, 243±19 nm, and 210±14 nm for COOH, OH, and CH₃ terminated SAMs. The difference in aspect ratio between CaP particles deposited on heterogeneous functionality surfaces is noticeable. CaP particles were 152±8 nm by 130±11 nm on the OH+COOH surface, 260±33 nm by 96±6 nm for the CH₃+COOH surface, and 248±37 nm by 88±5 nm for the OH+CH₃ surface.

The reason for coatings of different morphologies formed on molecularly heterogeneous surfaces in this experiment is not clear to us. We are also unaware of any report that presents structures of CaP coatings similar to those shown in FIGS. 2a and 2b. Possibilities for explanation could include a natural segregation of precipitated particles from solution on different surfaces due to different magnitude and nature of particle-surface interactions. Also, growth of particles could progress on homogeneous surfaces at the expense of dissolution of particles deposited on heterogeneous surfaces. Our experimental techniques cannot support or reject either of these two mechanisms.

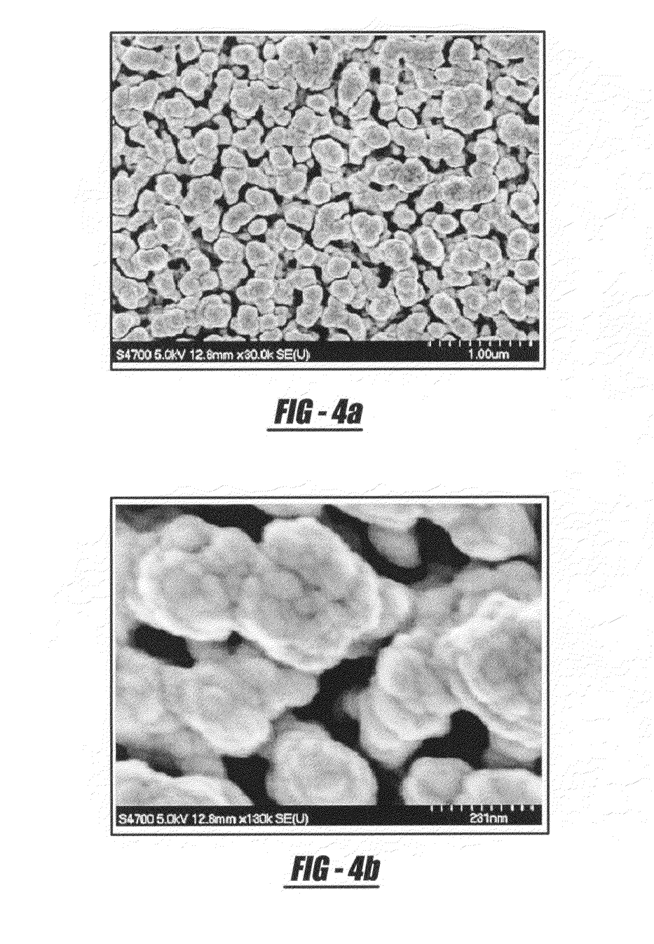

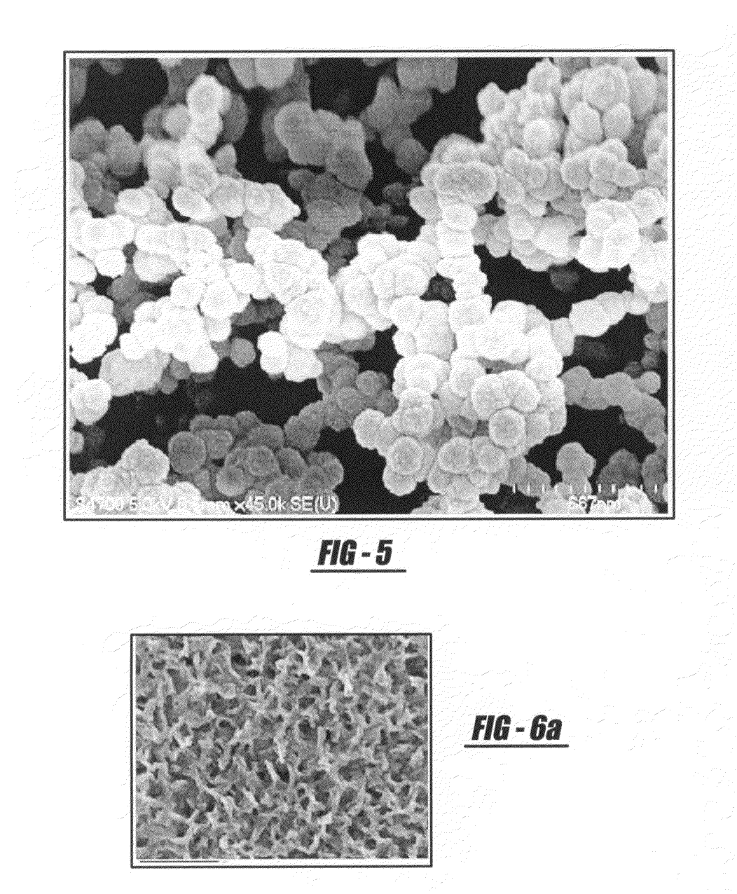

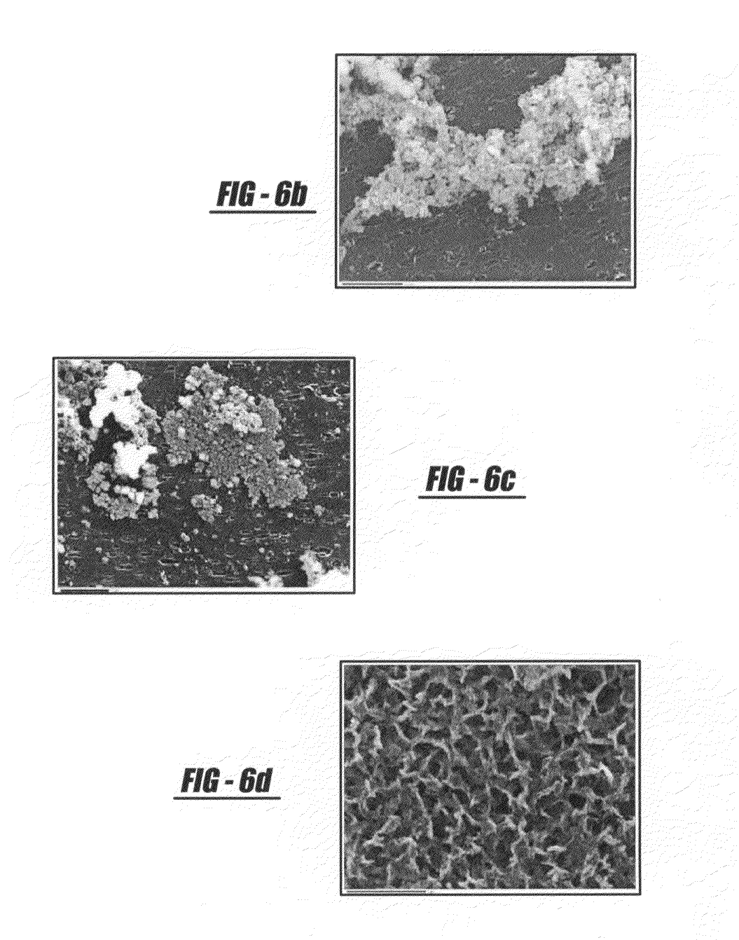

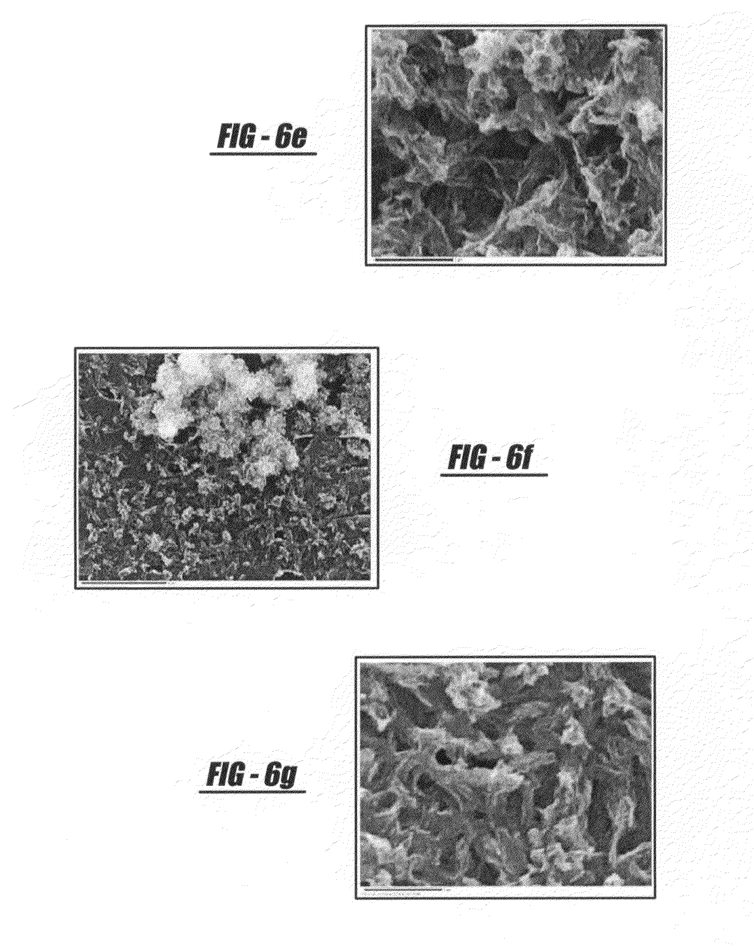

Further examination of CaP films on COOH-terminated substrates by FE-SEM highlighted the details of coating morphology as shown in FIGS. 4a and 4b. The spherical particles deposited on the COOH monolayer by Type I immersion show an underlying structure that resembles the clustering of even smaller 10-20 nm particles. It was determined that this structure is similar to that for particles separated from the SCPS solution as shown in FIG. 5. However, the particles, or aggregates of nanoparticles, separated from solution seem to be smaller, 169±5 nm. This difference may be a result of different batches of solutions used in coating deposition and preparation of precipitates, containing particles of different sizes. It can also suggest that indeed the aggregates of CaP coating grew to larger dimensions during the deposition experiment.

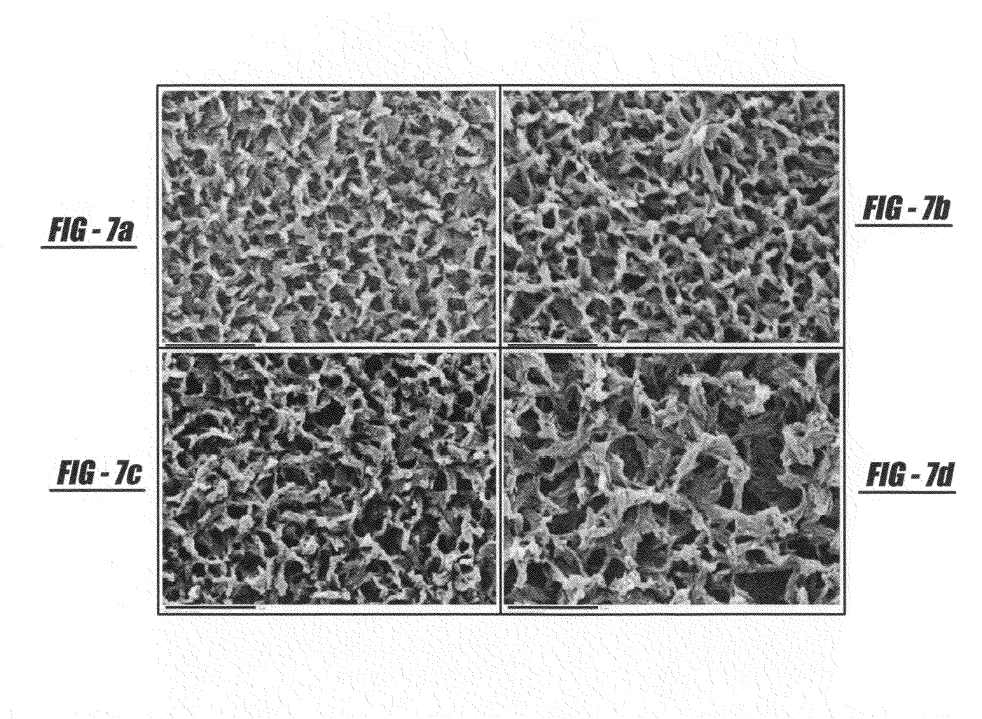

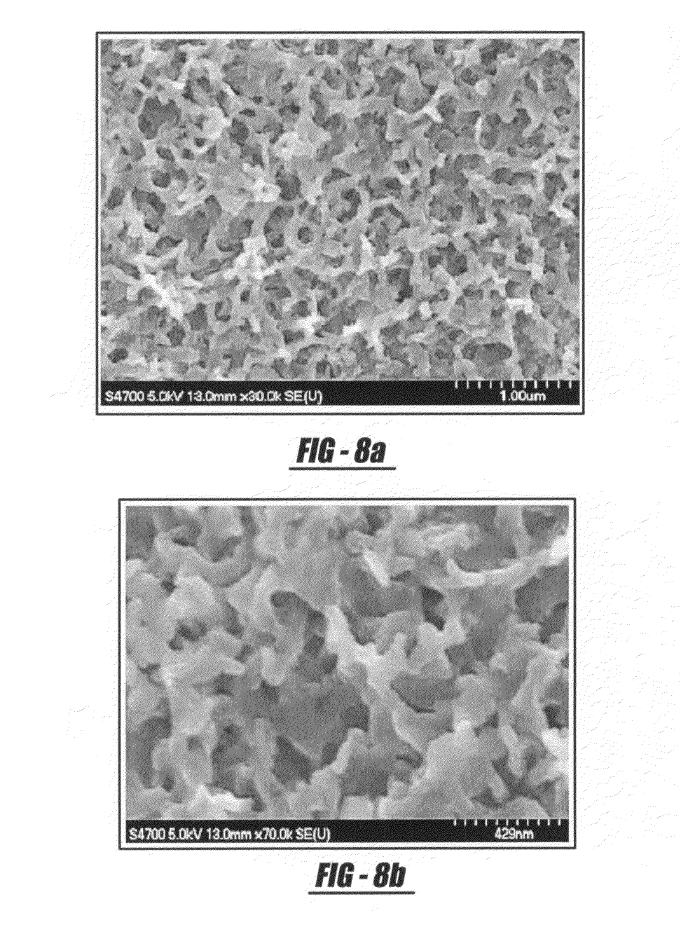

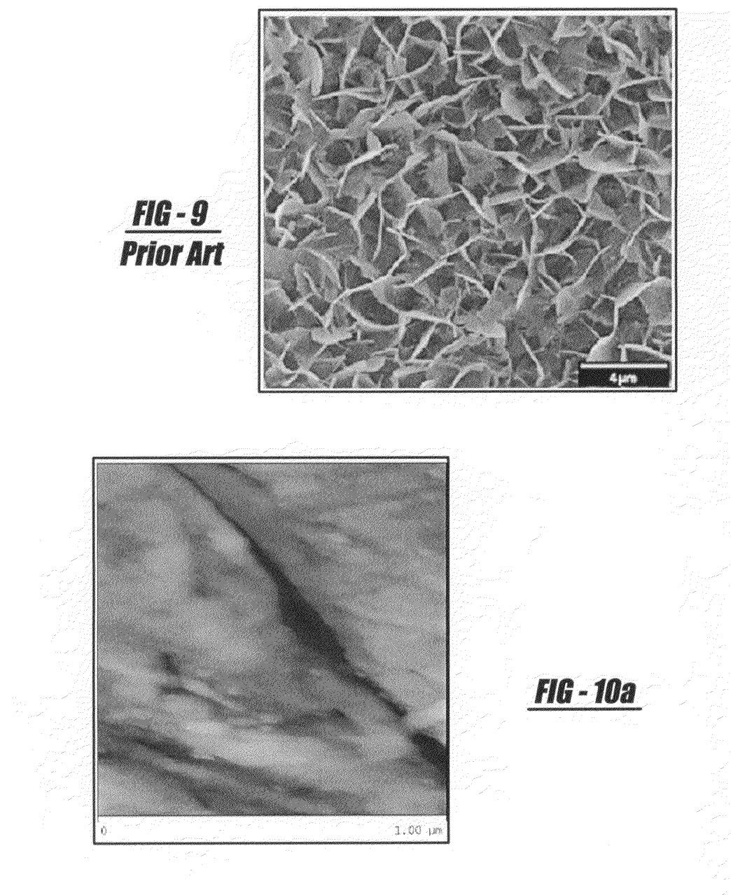

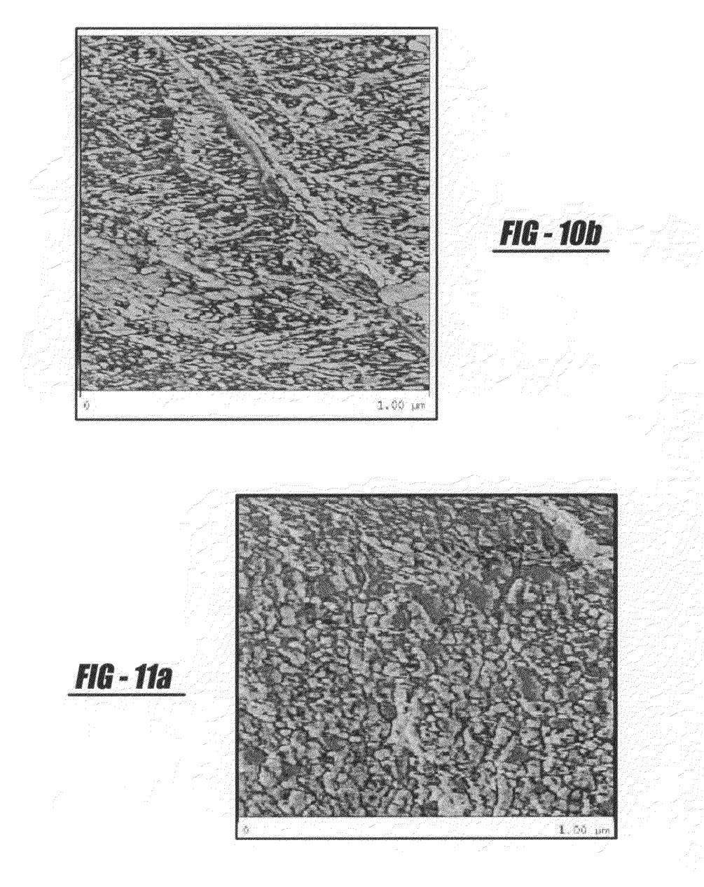

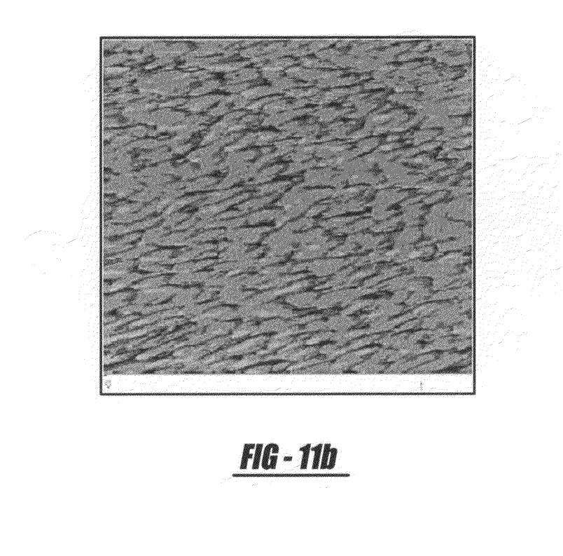

Type II Immersion Experiment. When Type II CaP deposition was used the CaP coating morphology was different as illustrated in FIGS. 6a-6g, FIGS. 7a-7d, and FIGS. 8a and 8b. Coatings did not reveal particle-type structures as in the case of coatings produced by Type I immersion method. The morphologies seem to be plate-like structures, but different from the “rose-like” morphology described by other researchers for CaP coatings deposited on metallic substra.^{10,27-29} For example, FIG. 9 shows the SEM image of a CaP coating prepared from a similar supersaturated solution on a titanium alloy substrate in our previous prior art studies.¹⁰ The plates of the CaP film deposited on the titanium alloy are very discrete and well-defined. In contrast, the plate structures of coatings deposited on the monolayers seem to have greater depth in all dimensions and are less discrete. Each of the plates seem to intertwine with neighboring plates as exhibited in FIGS. 7a-7d and FIGS. 8a-8b.

It is hypothesized that the differences in coating morphology of CaP film formation between the immersion techniques are due mainly to differences in conditions during immersion of substrates in the SCPS. As discussed earlier, in the case of Type I SCPS immersion experiments, the spherical morphology of the particles as illustrated in FIGS. 3a-3f and FIGS. 4a and 4b seemed to indicate precipitation of particles from solution and the subsequent build-up of CaP clusters as the dominant mechanism. Indeed, the similarity between particle morphologies on SAM surfaces deposited via Type I immersion and morphologies of particles separated from solution seem to support this hypothesis. In Type II immersion experiments the coating morphology as illustrated in FIGS. 6a-6g, FIGS. 7a-7d, and FIGS. 8a and 8b, is indicative of a heterogeneous nucleation and subsequent crystal growth mechanism. The porous nature of this particular morphology appears to resemble that of human bone and would allow for cellular infiltration of the structure and may enhance osteoblast adhesion when compared to the spherical morphologies.³⁰⁻³¹

### Porosity of Calcium Phosphate Coatings

Void fraction of the coatings was examined in regions representative of particle morphologies. This parameter was determined from 2-dimensional SEM images and should not be mistaken with porosity, although is meant to characterize the overall morphology of the CaP films. It should also not be confused with surface area coverage that is described hereinafter. Void fraction of the coatings varied greatly between the surface functionalities. Evenly dispersed voids and 12.2% void fraction was noted on the carboxyl-terminated monolayer. Concentrated open spacing comprising 13.9% of surface area shown in the image was observed on methyl-terminated monolayers. Evenly dispersed openings and a limited 8.8% void fraction was calculated for the hydroxyl-terminated monolayer.

CaP films on heterogeneous functionality SAMs showed characteristics exhibited by each of the homogeneous functionality components used to produce the surface. For instance, dispersion of pores on the heterogeneous OH(1)+COOH(1) functional surface was less concentrated than those observed on homogeneous OH surfaces, but also less evenly distributed than those observed on homogeneous COOH surface. Open space was greatest on CH₃+COOH (12.9%) and OH+COOH (12.1%) surfaces. Limited void fraction (7.1%) was observed for the OH+CH₃ sample.

In implementing Type IIA immersion for heterogeneous OH+COOH monolayers with varying OH:COOH ratio, no clear relationship was found between increasing amounts of hydroxyl-terminated thiols in solution and porosity of the deposited CaP films. OH(4)+COOH(1) surfaces produced CaP films with void fraction accounting for 22.7% of the surface area shown in FIG. 7C. OH(1)+COOH(2) surfaces elicited CaP films with void fraction making up 19.8% of the surface area shown in FIG. 7a. OH(2)+COOH(1) and OH(10)+COOH(1) surfaces produced films with the least amount of void fraction, 16.1% and 15.8%, respectively.

### Coverage of Substrate by Calcium Phosphate Coating

A uniform coating was produced on COOH and OH(1)+COOH(1) as illustrated in FIGS. 6a-6g terminated SAMs using Type II immersion. OH+COOH+CH₃ terminated SAMs preliminarily showed excellent coverage when examined by optical microscopy. Closer observation with SEM showed a very thin CaP layer on the substrate, but with nearly all of the surface area covered. OH terminated SAMs lacked a continuous CaP film. Small agglomerations could be seen all over the surface. The agglomerations were also found on CH₃ terminated SAMs, but small areas of growth were discovered beneath these clusters as shown in FIG. 6C. These growth areas coalesced with growth areas of neighboring agglomerations in some instances to begin the formation of a continuous CaP film. Heterogeneous functionality OH+CH₃ substrates exhibited even more coalescence of growth areas suggesting that chemical functionality may strongly influence the induction time of CaP formation.

Despite the differences in coating morphology, trends in substrate coverage were similar regardless of the immersion technique implemented. Substrate coverage of 100% was achieved on COOH, OH+COOH and OH+COOH+CH₃-terminated SAMs. As stated previously though, the coatings on the OH+COOH+CH₃ terminated SAM were extremely thin. CH₃+COOH terminated SAM substrates were approximately 80-85% covered with a CaP film. Heterogeneous functionality OH+CH₃ substrates elicited an approximate surface area coverage of 35-40%. Homogeneous CH₃ and OH substrates had the least amount of substrate coverage with 15% and 10%, respectively.

Varying the ratio of OH to COOH terminal sites on the SAMs had little effect on particle size and morphology as shown in FIGS. 8a and 8b. Porosity of the coatings was slightly different between ratios. It was observed that an increasing amount of OH groups did reduce the substrate coverage and uniformity of the CaP films.

### Calcium-to-Phosphorus Ratio in Coating

Calcium to phosphorus ratios of CaP films formed on self-assembled monolayers of differing functionalities varied for coatings produced in different experiments (as shown in Table 2).

TABLE 2
Calcium to phosphorus ratios of CaP coatings as a function of surface
functionality and immersion technique

| | Ca:P Ratio | | Immersion |
|---|---|---|---|
| Surface Functionality | (weight %) | (atomic %) | Technique |
| COOH | 2.00 ± 0.6 | 1.53 ± 0.5 | I |
| | 1.83 ± 0.1 | 1.42 ± 0.1 | IIB |
| OH | 2.94 ± 0.9 | 2.25 ± 0.7 | I |
| | 1.74 ± 0.1 | 1.34 ± 0.1 | IIB |
| CH₃ | 2.19 ± 1.2 | 1.66 ± 0.9 | I |
| | 1.71 ± 0.1 | 1.32 ± 0.1 | IIB |
| OH + COOH (1:1) | 1.96 ± 0.1 | 1.51 ± 0.1 | I |
| | 1.80 ± 0.1 | 1.40 ± 0.1 | IIB |
| OH + COOH (1:2) | 1.85 ± 0.2 | 1.43 ± 0.2 | IIA |
| OH + COOH (2:1) | 2.37 ± 0.4 | 1.83 ± 0.3 | IIA |
| OH + COOH (4:1) | 2.22 ± 0.1 | 1.71 ± 0.1 | IIA |
| OH + COOH (10:1) | 3.45 ± 1.2 | 2.67 ± 0.9 | IIA |
| CH₃ + COOH (1:1) | 2.03 ± 0.1 | 1.57 ± 0.1 | I |
| | 1.99 ± 0.2 | 1.54 ± 0.1 | IIB |
| CH₃ + OH (1:1) | 3.42 ± 1.9 | 2.64 ± 1.5 | I |
| | 1.77 ± 0.1 | 1.37 ± 0.4 | IIB |
| CH₃ + COOH + OH (1:1:1) | 1.76 ± 0.1 | 1.34 ± 0.1 | IIA |

When Type I immersion was employed, the coatings were very calcium-rich. However, when Type IIB immersion was used, the films were calcium-deficient, with the overall Ca:P ratio lower than that of stoichiometric hydroxyapatite. This difference in coating compositions may highlight the difference in the underlying mechanism of deposition between the two immersion methods. The calcium-rich nature of the films, similarity in morphology to CaP particles separated from solution and the charge number of calcium ions (2+) compared to phosphate complexes (1−) imply that Type I immersion relies on an electrostatic interaction for the deposition of CaP films.

It is also worth noting that hydroxyl-dominated SAMs and SAMs with an equal ratio of hydroxyl and other groups elicited the most calcium-rich coatings when Type I and Type IIA immersion was employed. When Type IIB immersion was used, hydroxyl containing SAMs had the lowest calcium to phosphorus ratios.

From this investigation of the effects of surface chemistry on nucleation, growth and adhesion of CaP films, it was found that the self-assembled monolayers (SAMs) of alkanethiols served as model organic substrates in the biomimetic deposition of CaP coatings from supersaturated calcium phosphate solutions. It was further found that homogeneous COOH-terminated SAMs and heterogeneous OH+COOH SAMs produced CaP coatings that covered 100% of the substrate surface area within three days of chemical immersion. SAMs containing a predominantly methyl, or hydroxyl character failed to elicit continuous CaP films covering all of the surface area. This trend of surface area coverage was observed regardless of the immersion method employed, which suggests that the deposition process is strongly dependent on surface functionality.

Both deposition methodology and substrate surface functionality had also influenced the chemical composition of the formed calcium phosphate coatings. Calcium to phosphorus ratios of CaP films varied from about 1.3 to 2.7 for coatings produced on SAMs of differing functionalities. OH-terminated SAMs induced formation of deposits with enriched calcium content whereas coatings with stoichiometry close to that for hydroxyapatite were preferentially formed on SAMs having COOH groups.

### Effect of Polyethylene Pretreatments on the Biomimetic Deposition and Adhesion of Calcium Phosphate Films

Having gained a clearer insight of the surface chemistry variables associated with the adhesion of CaP films in a biomimetic deposition process it now became necessary to apply this learned knowledge to a substrate of choice.

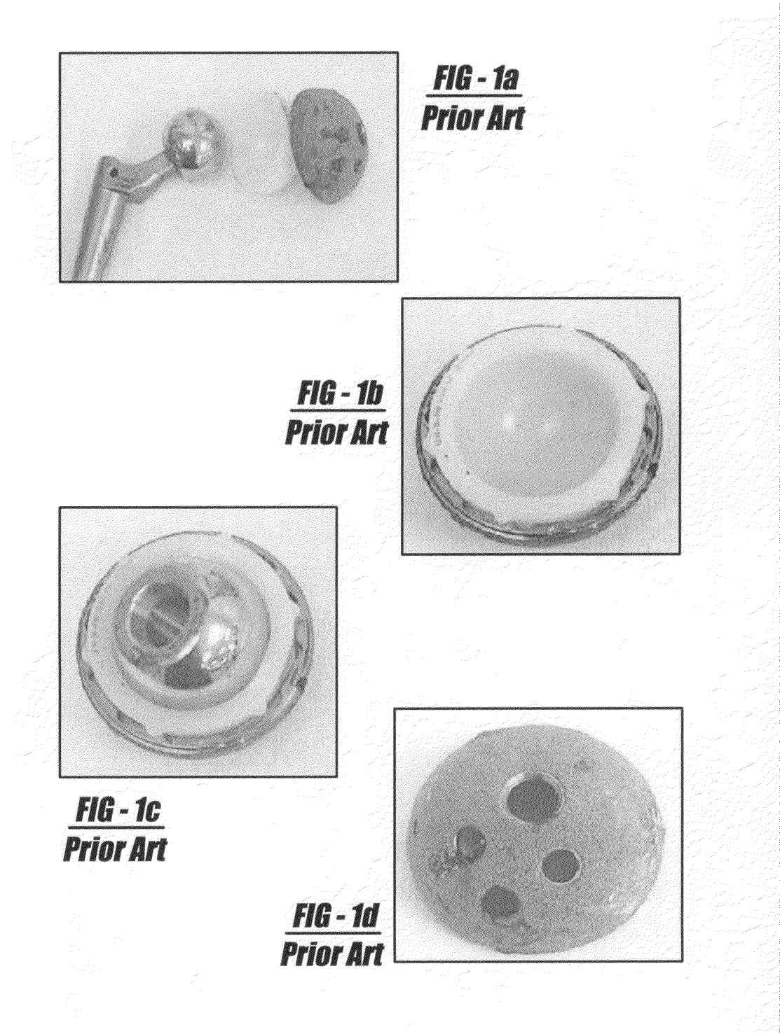

With elastic properties similar to biological tissue found in joint capsules and the ability to achieve a smooth surface through common machining techniques, polyethylene has gained wide-spread support as a material of choice for bearing surfaces in total shoulder, knee and hip arthroplasty.³² Most polyethylene components used in joint replacements are equipped with a metal component such as the tibial tray in total knee arthroplasty, or the acetabular cup in total hip arthroplasty shown in FIG. 1. The metal devices contain and stabilize the polyethylene bearing surface and act as the interface between bone and the implant. However, these metal-backed systems have been shown to generate polymeric wear debris through a process known as backside-wear.³³⁻³⁴ Polyethylene wear debris elicits an inflammatory response from the host system and can lead to implant loosening by osteolysis.³⁵⁻³⁶ Developing a method by which polyethylene components can bond directly to bone would eliminate the need for the aforementioned metallic containment devices. This bonding would also reduce the need for PMMA-based bone cements, which have been shown to have deleterious biological effects due to the extremely exothermic nature of the cement curing process.⁷⁻⁹

As stated above, bioactive calcium-phosphate (CaP) coatings have been employed in several orthopedic applications to provide an environment conducive to bone growth at the surface of implants.^{10-16,19,37} Plasma sprayed hydroxyapatite (HA) is the most common form of bioactive coating used to enhance hard tissue integration with orthopedic implants. Since the plasma spray deposition method is a line of sight process, coatings produced on implants with complex geometries (screws, hip stems) are often non-uniform in terms of substrate coverage and coating thickness.¹⁰ The plasma spray deposition process also entails the use of high temperatures which can lead to heterogeneous coating properties and increased crystal sizes. Increased crystal sizes effectively reduce nano-scale surface roughness, which has been shown to negatively impact the state of differentiation of osteoblast cells.³⁸ Chemical immersion biomimetic processes have therefore gained favor with the use of polyethylene substrates. Coating the non-bearing surface of polyethylene components with a bone-like, biomimetic CaP film by a similar method would increase the integration of the device with the patient's surrounding hard tissue (osseointegration).^{10,28,30,37,39-44} Through the process of osseointegration, the polyethylene would be integrated into the structure of the hard tissue thereby imparting long-term stability of the device in vivo. A direct benefit of this could be the elimination of devices commonly used in conjunction with the polyethylene bearing components, thereby reducing the mass and number of components to be implanted. As mentioned previously, this would also reduce the generation of wear debris in the joint capsule.

A major challenge facing the deposition of biomimetic CaP films on polyethylene substrates is the adhesion characteristics between the film and substrate. Adhesion between metallic substrates and CaP films have been shown to be much higher when compared to polymeric substrates.^{11-12,19} Kokubo and others have been successful in applying substrate pretreatments to polymeric materials to improve the adhesion characteristics of biomimetic CaP films, but details of the effects of these substrate pretreatments are not deeply understood.^{11-12,19}

In this investigation, extruded polyethylene tensile bars were sectioned, cleaned and characterized using contact angle analysis and atomic force microscopy (AFM). Samples were then subjected to UV irradiation, or glow discharge processing for varying amounts of time. The effects of these pretreatments on the surface properties of the polyethylene samples were investigated using contact angle analysis and AFM. Samples were then immersed in a supersaturated calcium phosphate solution for deposition of a biomimetic CaP film. Nano-scratch testing was performed on the CaP-coated polyethylene samples to examine the effects of the substrate pretreatments on substrate-coating adhesion characteristics. Scanning electron microscopy was used to evaluate the morphology of the resulting coating. Fourier transform infrared spectroscopy (FTIR), energy dispersive spectroscopy (EDS) and X-ray diffraction (XRD) were used to evaluate the composition and structure of the coatings.

### Experimental Procedure

### Sample Preparation and Substrate Treatment

Tensile specimens of commercial high density polyethylene (available from Dow Chemical Co., PE) were sectioned along the neck with a silicon carbide cutting wheel to yield 12.5×12.5 mm samples. The edges were smoothed and extraneous material was removed using a surgical scalpel. Samples then underwent 5 minutes of ultrasonic cleaning in methanol (histological grade), surfactant solution (Micro 90, from International Products Inc.) and deionized water (18MΩ). The PE samples were then dried in an L-C oven for 10 minutes at 75° C.

### Substrate Pretreatments

### UV Irradiation Pretreatment

Three cleaned PE samples were placed in a UV irradiation chamber (available from BioForce Laboratories Inc.) for 10, 30 or 60 minutes. Samples were placed approximately 50 mm away from the light source and were subjected to UV irradiation with main wavelengths of 184.9 and 253.9 nm.

### Glow Discharge Pretreatment

PE samples were subjected to radio-frequency driven glow discharge processing (Jupiter II Reactive Ion Etcher, March Instruments Inc.) for 10, 30, 60 or 300 seconds under an O₂ environment with a gas flow of 25 cm³/s at a pressure of 186 mTorr. A forward power of 50 Watts and a frequency of 13.56 MHz were used to process the samples for the varying time.

### Substrate Characterization

### Atomic Force Microscopy

Atomic force microscopy was used to image untreated PE substrates and to investigate any topographical and structural changes brought about by the surface pretreatments. Silicon cantilevers (˜300 kHz, ˜40N/m) were used for sample surface scanning in Tapping Mode. Scan sizes of 1 μm² were used for characterization of surface roughness and phase differences. At least three scans were performed on each untreated and treated PE surface to gather information representative of each of the surfaces.

### Contact Angle Measurements

The polyethylene substrates were characterized by measurements of advancing and receding contact angles performed by implementation of the sessile drop technique.²² A Kruss-G10 goniometer with drop shape analysis software was used for measuring seven advancing and seven receding contact angles of de-ionized water, ethylene glycol (HOCH₂CH₂OH, MW=62.07 g/mol, 99+%, Fisher Scientific), glycerine (C₃H₈O₃, MW=92.09 g/mol, Fisher Scientific) and diiodomethane (CH₂I₂, MW=267.84 g/mol, J.T. Baker Inc.).

The surface tension and surface tension components of the substrates were calculated using the semi-empirical Lifshitz-van der Waals Lewis acid-base interaction model developed by van Oss, Good, and Chaudhury.⁴⁵ This model asserts that the surface tension of a material is the sum of the Lifshitz-van der Waals components of surface energy and the geometric mean of the electron acceptor and donor parameter of the material.

### Calcium-Phosphate Deposition

A supersaturated calcium-phosphate solution (SCPS) was prepared in accordance with previously described methods by combining 5.6 mM CaCl₂.2H₂O (MW=147.02 g/mol, from Fisher Scientific) and 3.34 mM NaH₂PO₄.H₂O (MW=137.99 g/mol, 99.4%, also available from Fisher Scientific) in 1:1 volumetric ratio.¹⁰ The resting pH of the solution was approximately 3.2 and a tris-buffer (tris(hydroxymethyl)-aminomethane, C₄H₁₁NO₃, MW=121.14 g/mol, 99+%, Aldrich) was used to elevate and maintain the solution pH to approximately 7.35. Solution pH was monitored during preparation (pH-Meter, available from Metrohm Inc.).

After substrate pretreatment, samples were immersed vertically in the SCPS. Deposition took place in a Pyrex dish filled with approximately 250 mL of SCPS, while the samples were held in a vertical position by painted metal clips. SCPS immersion lasted for three days with daily solution refreshments. Solution refreshments were performed by pipetting the old solution from the container and gently pouring in fresh solution, so as to not disturb the samples. On the final day of immersion, samples were removed from the SCPS, rinsed with DI water and allowed to dry in an L-C oven at 75° C. for 10 minutes.

### Coating Characterization

### Morphology and Substrate Coverage

CaP morphology was examined using the JEOL JSM-6400 scanning electron microscope (SEM) with an accelerating voltage of 20 kV. Cap-coated PE samples were sputtered with 2.5 nm of carbon to improve SEM image quality and reduce charging effects.

The SEM images were analyzed using Scion Image 4.02 (available from Scion Corporation) to gather information about the size of CaP particles/structures in the coatings.

### Chemical and Structural Composition

Energy dispersive x-ray spectrometry (EDS; JEOL JSM-6400) was used to elicit information regarding the calcium to phosphorus ratio of the CaP coatings. Spectra were gathered from five different locations on the coating and quantitative analysis was performed using Spirit software.

X-ray diffraction (XRD, Scintag XDS 2000 Diffractometer) was employed to gather information about the structure and composition of the deposited CaP film. An untreated PE sample underwent immersion in SCPS for five days with daily solution refreshments to deposit enough material for this test. The sample was then allowed to dry in an L-C oven for 1 hour at 75° C. The CaP-coated PE sample was then subjected to an 18-hour XRD scan utilizing Cu—K_{α} radiation (λ=1.54 Å). The Bragg-Brentano diffractometer was used in the θ-2θ configuration.

The 5-day coating composition was also analyzed by Fourier transform infrared spectroscopy (FTIR, Spectrum AssureID, obtained from Perkin-Elmer Inc.). An attenuated total reflectance (ATR) attachment was added to the FTIR to permit analysis of the coating.

### Coating-Substrate Adhesion

A nano-indenter system (Nanoindenter XPS, from MTS Inc.) equipped with a Berkovich-style scratch tip was used for interrogation of coating-substrate adhesion parameters via scratch testing. The Berkovich tip was ramped through the coating with an applied normal load range of 0-20 mN and a scratch length of 500 μm. A single batch of ten scratches was performed on each sample. Scratches were spaced 100 μm apart to sample a representative area of the coating.

Scratches on each of the samples were examined under SEM. Images of all ten scratches, and groups of three scratches within the batch were gathered at an accelerating voltage of 20 kV, and at a magnification of 55× and 250× respectively. Digital image analysis software (Scion Image, from Scion Corporation) was used to digitally measure the distance from the point of initial load application to the point of coating delamination for each scratch. The length was then used to determine the applied normal load and lateral force (applied lateral force perpendicular to the scratch path) by correlating the position at which delamination occurred with the position of the scratch tip. After determining the position of the scratch tip, the force parameters were located in the data file generated and recorded during the scratch tests. All scratch lengths were measured to ensure that only the 500 μm prescribed length of the scratch was taken into account.

### Results and Discussion

### Effect of Surface Treatment on Topography of Polyethylene Substrates

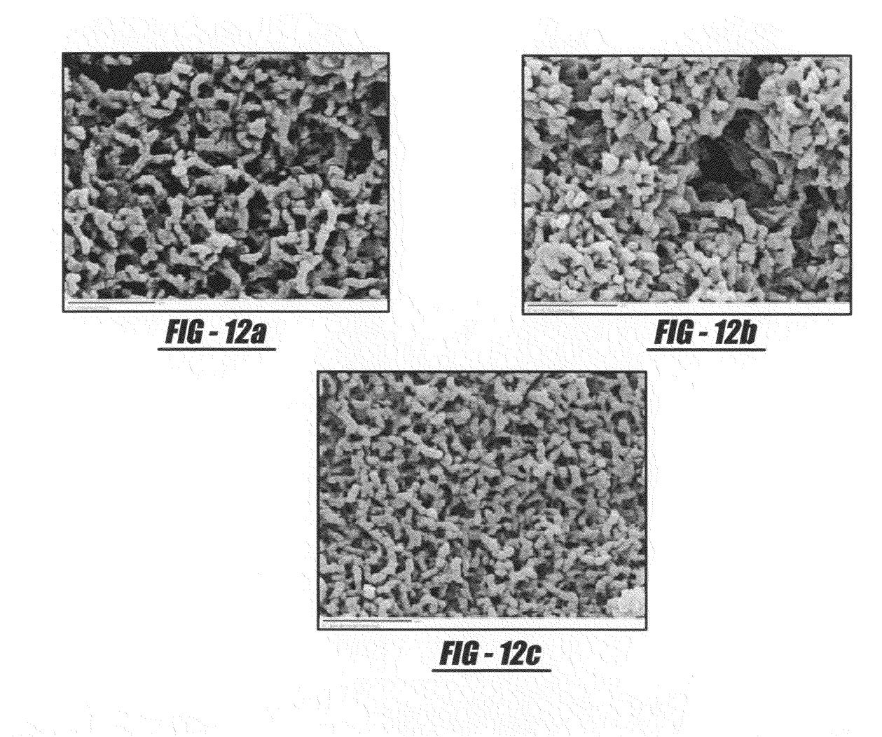

As a basis for comparison, 1 μm² AFM scans were performed on a cleaned, untreated polyethylene substrate. FIG. 10a illustrates the topography of the untreated polyethylene surface. FIG. 10b is a phase image which depicts the differences in elastic properties between various domains of the polymer surface and their different adhesion characteristics in contact with the silicon AFM cantilever tip. This image, as the name implies, maps the different phases present in a sample. As can be seen from the image, there exists an amorphous (dark) and crystalline (bright) phase in the PE sample, with well-defined boundaries.

AFM phase images illustrated in FIGS. 11a and 11b show that UV irradiation and glow discharge pretreatments seem to preferentially attack amorphous regions of the polyethylene samples. It is also well-documented that specific treatments of polyethylene increase the degree of cross-linking in the polymer.⁴⁶⁻⁴⁷ Increases in cross-link density would shrink the size and distribution of amorphous domains in the sample, since regions of high cross-linking would have elastic behavior similar to that of purely crystalline domains.

UV irradiation and glow discharge processing yielded block-shaped domains at the surface of the PE samples. The block-shaped regions were larger and more pronounced on the glow discharge treated samples when compared to the UV treated samples.

Analysis of the roughness parameters ascertained by AFM and summarized in Table 3, below, suggest little, if any, effect of substrate pretreatment on surface topography. There is a slight drop in the roughness parameter (ratio between the actual surface area and the projected surface area of the scan) noted for samples having undergone glow discharge pretreatments. Geometric roughness (RMS) and average roughness (Rₐ) remain unchanged, for the most part, throughout the different oxidation pretreatments.

TABLE 3
Roughness characteristics of untreated and treated 1 × 1 μm² PE
surfaces obtained with AFM.

| Type of | | | | |
|---|---|---|---|---|
| Substrate | Pretreatment | RMS | Rₐ | r |
| Pretreatment | Conditions | (nm) | (nm) | (—) |
| Control | None | 7.4 ± 2.9 | 5.7 ± 2.1 | 1.14 ± 0.05 |
| UV | 10 min. | 8.9 ± 4.7 | 6.6 ± 3.2 | 1.10 ± 0.02 |
| Irradiation | 30 min. | 4.6 ± 0.4 | 3.5 ± 0.3 | 1.10 ± 0.02 |
| | 60 min. | 5.2 ± 3.1 | 4.0 ± 2.3 | 1.13 ± 0.06 |
| Glow | 50 W 10 s. | 6.0 ± 0.2 | 4.7 ± 0.1 | 1.05 ± 0.01 |
| Discharge | 50 W 30 s. | 11.7 ± 2.4 | 9.4 ± 1.0 | 1.37 ± 0.07 |
| | 50 W 60 s. | 8.1 ± 1.1 | 6.4 ± 1.0 | 1.06 ± 0.03 |
| | 50 W 300 s. | 6.7 ± 1.7 | 5.3 ± 1.3 | 1.06 ± 0.00 |

### Contact Angle and Surface Energy Analysis

Table 4 summarizes the advancing and receding contact angles, along with the contact angle hysteresis (difference between advancing and receding contact angles) for untreated and treated PE surfaces.

TABLE 4
Advancing (θ_{Adv.}) and receding (θ_{Rec.}) contact angles and contact
angle hysteresis (Δθ) for untreated and pretreated polyethylene substrates.

| Type of | | Contact Angle (degrees) | | | | |
|---|---|---|---|---|---|---|
| Substrate | Pretreatment | | | Ethylene | | |
| Pretreatment | Conditions | | Water | Glycol | Glycerine | Diiodomethane |
| Control | | θ_{Adv.} | 85.9 ± 1.5 | 68.5 ± 2.0 | N/A | 49.1 ± 1.5 |
| | | θ_{Rec.} | 53.8 ± 1.2 | 36.1 ± 3.7 | N/A | 29.3 ± 3.6 |
| | | Δθ | 32.1 | 32.4 | N/A | 19.8 |
| UV | 10 min. | θ_{Adv.} | 71.7 ± 1.3 | 53.0 ± 0.8 | N/A | 46.1 ± 0.6 |
| Irradiation | | θ_{Rec.} | 47.7 ± 1.9 | 19.9 ± 3.2 | N/A | 15.6 ± 1.1 |
| | | Δθ | 24.0 | 33.1 | N/A | 30.5 |
| | 30 min. | θ_{Adv.} | 78.6 ± 1.9 | 41.2 ± 1.5 | N/A | 44.4 ± 1.0 |
| | | θ_{Rec.} | 35.0 ± 1.8 | 11.7 ± 1.2 | N/A | 12.9 ± 1.5 |
| | | Δθ | 43.6 | 29.5 | N/A | 31.5 |
| | 60 min. | θ_{Adv.} | 75.7 ± 1.2 | 42.8 ± 2.4 | N/A | 45.8 ± 2.4 |
| | | θ_{Rec.} | 38.0 ± 2.0 | 15.0 ± 1.3 | N/A | 17.5 ± 1.6 |
| | | Δθ | 37.7 | 27.8 | N/A | 28.3 |
| Glow | 50 W 10 sec. | θ_{Adv.} | 51.1 ± 2.2 | N/A | 57.3 ± 0.6 | 41.5 ± 0.9 |
| Discharge | | θ_{Rec.} | <5 | N/A | N/A | <5 |
| | | Δθ | 46.1 | N/A | N/A | 36.5 |
| | 50 W 30 sec. | θ_{Adv.} | 47.4 ± 1.4 | N/A | 52.3 ± 1.9 | 45.2 ± 0.6 |
| | | θ_{Rec.} | <5 | N/A | N/A | <5 |
| | | Δθ | 42.4 | N/A | N/A | 40.2 |
| | 50 W 60 sec. | θ_{Adv.} | 47.3 ± 1.1 | N/A | 52.4 ± 2.4 | 43.3 ± 0.9 |
| | | θ_{Rec.} | <5 | N/A | N/A | <5 |
| | | Δθ | 42.3 | N/A | N/A | 38.3 |
| | 50 W 300 sec. | θ_{Adv.} | 48.8 ± 0.8 | N/A | 53.4 ± 1.0 | 42.2 ± 1.5 |
| | | θ_{Rec.} | <5 | N/A | N/A | <5 |
| | | Δθ | 43.8 | N/A | N/A | 47.2 |

Contact angle hysteresis values and low standard deviations for most samples imply that the surfaces are of good quality and the results are reproducible. Non-uniform etching and oxidation of polymer are expected to produce irreproducible surfaces of different heterogeneity pattern.

Advancing water contact angles of nearly 90° for untreated PE samples were observed and are indicative of a hydrophobic material. The values are lower by 5-15 degrees than what is usually reported for polyethylene in literature.^{22,48} Exposure of the samples to UV irradiation or glow discharge changed the hydrophobic character of the polymer to a more hydrophilic character, as evidenced by a reduction in the advancing contact angle values measured for water drops. The production of surface polar groups (carboxyl, hydroxyl, carbonyl, esters, etc.) upon physico-chemical pretreatment is well-documented.^{11-12,19}

As can be seen in Table 4, above, glow-discharge processing provided the most dramatic reduction in advancing water contact angles. As is the case for all treatments, a saturation point in the reduction of contact angles was observed. This saturation in contact angle reduction may be related to the achievable number of polar groups at the sample surface by particular treatment technique. Overall, UV irradiation had the least severe impact on advancing water contact angles.

Samples having undergone a glow discharge pretreatment with a forward power of 50 W were shown to have the highest total surface energy and the highest electron donor (γ⁻) parameter as shown in Table 5, below. The γ⁻ parameter is defined as a quantification of the active sites that attract electrons from another material, in terms of surface energy. This attraction and sharing of electrons may not only aid in improving substrate coverage by biomimetic CaP films, but it may also contribute to the enhancement of adhesion of these films to the polymeric substrate.

TABLE 5
Surface energy and surface energy components of untreated and treated
polyethylene substrates.

| Type of | | | | | |
|---|---|---|---|---|---|
| Substrate | Pretreatment | Surface Energy and Surface Energy Components (mJ/m²) | | | |
| Pretreatment | Conditions | γ^{LW} | γ⁺ | γ⁻ | γ^{Total} |
| Control | None | 34.80 ± 0.82 | 0.15 ± 0.12 | 7.03 ± 1.49 | 32.77 ± 1.19 |
| UV | 10 min. | 36.41 ± 0.36 | 0.01 ± 0.01 | 14.38 ± 1.55 | 37.18 ± 0.64 |
| Irradiation | 30 min. | 37.30 ± 0.01 | 0.84 ± 0.01 | 4.27 ± 0.01 | 41.10 ± 0.01 |
| | 60 min. | 36.60 ± 0.01 | 0.63 ± 0.01 | 6.78 ± 0.01 | 40.70 ± 0.01 |
| Glow | 50 W 10 s. | 38.8 | 0.04 | 33.31 | 41.2 |
| Discharge | 50 W 30 s. | 36.9 | 0.36 | 34.51 | 43.9 |
| | 50 W 60 s. | 36.9 | 0.35 | 34.66 | 43.9 |
| | 50 W 300 s. | 38.5 | 0.21 | 33.15 | 43.8 |

### Substrate Coverage and CaP Coating Morphology

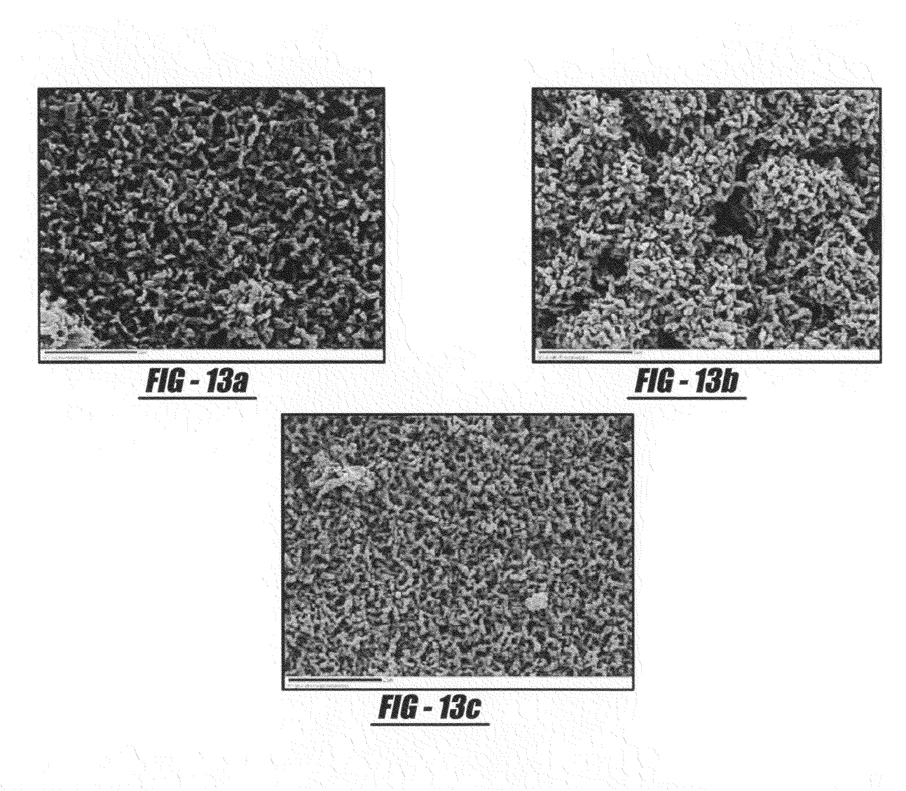

The appearance of spherical CaP particle morphologies first noted during the self-assembled monolayer (SAM) study, Type I immersion experiment shown in FIGS. 3a-3f, were once again observed on treated and untreated PE substrates. FIGS. 12a-12c and FIGS. 13a-13c show SEM micrographs of the CaP particle and cluster morphologies on the treated and untreated PE substrata. The underlying structures of the particles shown in FIGS. 12a-12c are several spherical particles with a diameter of 85.0 nm (±12.3 nm) clustered together. The clustering of the spherical particles gives the impression of an elongated structure. The dimensions of the elongated clusters are 140.0×96.9 nm (±20.8 nm, ±10.3 nm), as determined by digital image analysis.

Particle and cluster morphologies were similar within each type of substrate pretreatment. Overall coating morphologies did vary between the different substrate pretreatment groups however. CaP clusters were evenly dispersed on the control, UV irradiated and glow discharge treated samples.

AFM analysis of the PE substrates before and after the prescribed pretreatments indicate little or no change in topography. This fact suggests that surface functionality plays a role in determining the deposition characteristics of CaP films on PE substrata. Similarities in the coating morphologies and deposition characteristics also suggest that the oxidation pretreatments aid in the production of chemically similar surface of the PE samples.

Substrate coverage was near 100% for all treated and untreated substrates. CaP films on the PE substrate treated with UV irradiation for 60 minutes showed an 80-85% coverage of the free surface area.

Near 100% substrate coverage of the untreated (control) PE sample suggests that some sort of surface functionality was preexisting. Results from the surface chemistry on nucleation, growth and adhesion on CaP films indicated that CaP nucleation and growth should be weak on surfaces possessing a dominant “methyl” (CH₃) character, such as would be the case for untreated PE substrates. Stearates had been incorporated into the polymer blend as mold release agents during PE processing at the Dow Corporation⁴⁹ and they are probably responsible for deposition of CaP films on untreated polyethylene samples.

### CaP Coating Composition

Substrate pretreatments were found to have little effect on the calcium to phosphorus ratios of the bone-like CaP films when this parameter was investigated by EDS as shown in Table 6, below. Irradiation of the PE substrates with UV light tends to produce a film with a lower Ca:P ratio. Increased time of exposure to the UV light intensified this effect.

TABLE 6
Calcium to phosphorus ratios of CaP films on untreated and treated polyethylene
substrates obtained by EDS.

| Type of Substrate | Pretreatment | | Calcium to Phosphorus Ratios | | | |
|---|---|---|---|---|---|---|
| Pretreatment | Conditions | | Weight % | Atomic % | Weight % | Atomic % |
| Control | None | | 1.80 ± 0.05 | 1.39 ± 0.04 | 1.78 ± 0.02 | 1.37 ± 0.02 |
| UV | 10 | min. | 1.77 ± 0.02 | 1.37 ± 0.02 | 1.73 ± 0.05 | 1.34 ± 0.04 |
| Irradiation | 30 | min. | 1.75 ± 0.05 | 1.35 ± 0.04 | | |
| | 60 | min. | 1.68 ± 0.04 | 1.30 ± 0.03 | | |
| Glow | 10 | s. | 1.84 ± 0.01 | 1.42 ± 0.01 | 1.81 ± 0.02 | 1.40 ± 0.01 |
| Discharge | 30 | s. | 1.81 ± 0.01 | 1.40 ± 0.01 | | |
| | 60 | s. | 1.79 ± 0.01 | 1.38 ± 0.01 | | |
| | 300 | s. | 1.81 ± 0.05 | 1.40 ± 0.04 | | |

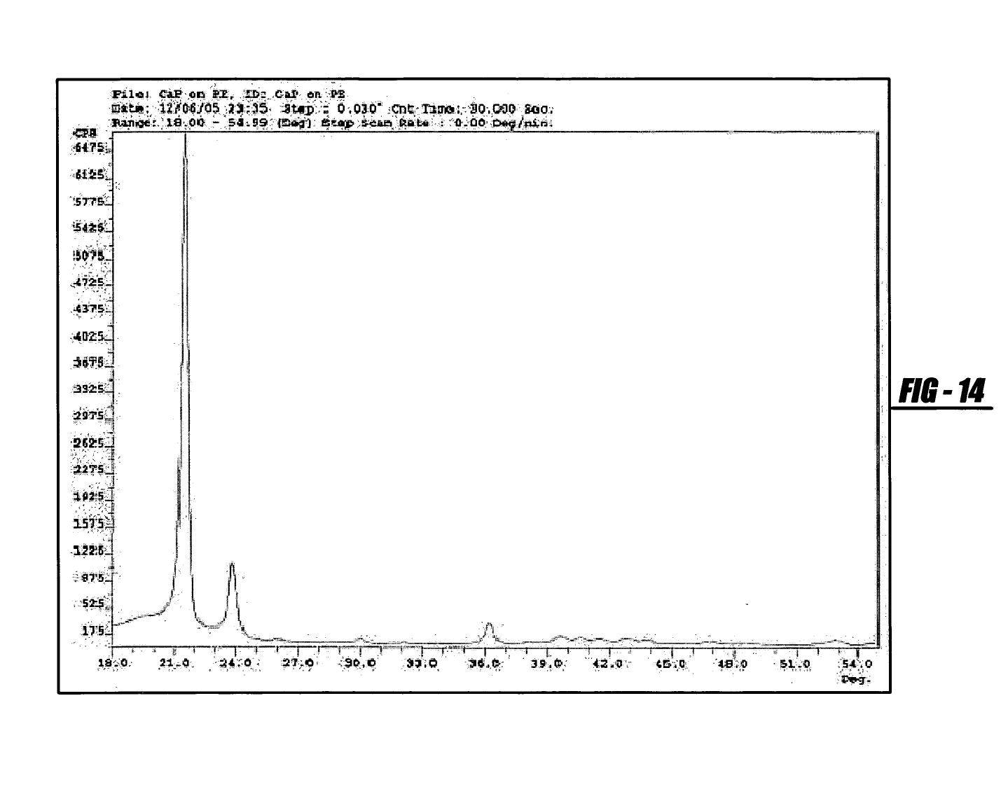

FIG. 14 exhibits an X-ray diffraction scan of the five day CaP coating on an untreated polyethylene substrate showed a spectrum indicative of most apatite coatings.^{10,29,50} Small, broad peaks were noted at 26°, 36°, 47° and 53° 2θ in FIG. 14. Fairly sharp peaks were observed at 21° and 24° 2θ and are attributed to the polyethylene substrate. Amorphous ridges are also present in the spectrum between 18 and 27° and between 39 and 44° 2θ. These are also attributed to the substrate.

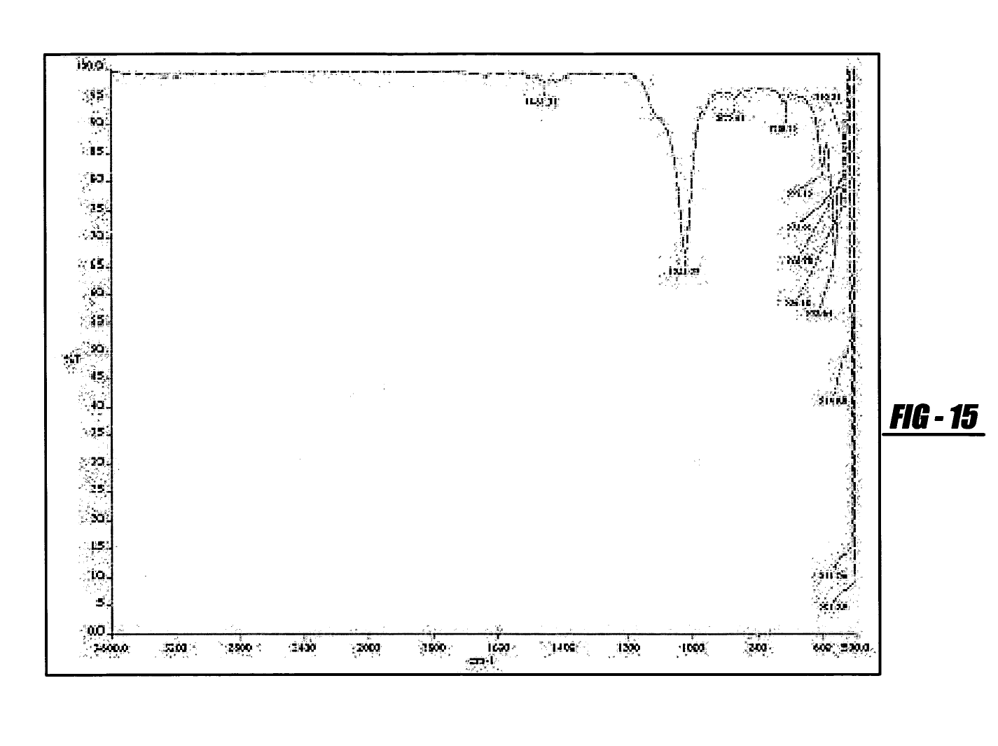

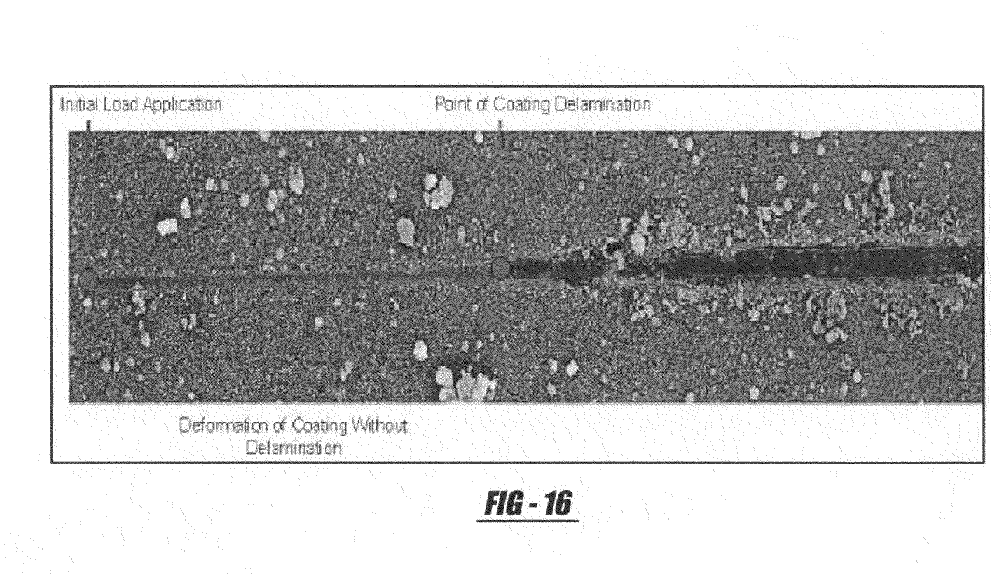

The infrared spectrum of the CaP coating on a PE substrate illustrated in FIG. 15 is also indicative of an apatite coating. Several peaks appear in the 400-1100 wavenumber range, which are characteristic of certain phosphate groups found within the structure of the CaP coating. Some of the more important peaks to note occur at 1023, 599 and 573 cm⁻¹. These peaks are characteristic of hydroxyapatite.^{10,50}

### Effects of Substrate Pretreatments on Adhesion of CaP Coatings

FIG. 16 illustrates an SEM micrograph of a scratch test performed on a CaP coated substrate. Points of initial load application were clearly defined by the beginning of localized deformation of CaP coating. Deformation of the coating became more pronounced along the scratch path until the point of delamination was reached. Deformation of the PE substrate was clearly visible directly after the point of coating delamination, which also aided in the determination of coating failure.

TABLE 7
Summary of critical normal loads, 95% confidence interval for the
critical normal load data obtained from nano-scratch tests of CaP-coated
treated and untreated PE substrates.

| Type of | | | Critical | 95% Confidence |
|---|---|---|---|---|
| Substrate | Pretreatment | | Normal | Interval for Critical |
| Pretreatment | Conditions | | Load (mN) | Normal Load (mN) |
| Control | None | | 5.5 ± 0.9 | 4.9–6.1 |
| UV Irradiation | 10 | min. | 5.8 ± 0.7 | 5.3–6.3 |
| | 30 | min. | 5.2 ± 0.5 | 4.8–5.6 |
| | 60 | min. | 4.3 ± 0.2 | 4.1–4.4 |
| 50 W Glow | 10 | s. | 8.6 ± 2.1 | 7.2–9.9 |
| Discharge | 30 | s. | 13.5 ± 2.0 | 12.1–15.0 |
| | 60 | s. | 10.6 ± 0.9 | 8.4–11.0 |
| | 300 | s. | 9.7 ± 2.1 | 10.0–11.2 |

Table 7, above, provides a summary of the critical normal loads obtained from nano-scratch tests of CaP coatings on untreated and treated PE substrata. The critical normal loads, while they provide little information regarding the substrate-coating adhesive strength in shear loading, do show a fairly clear relationship based on substrate pretreatments. These normal loads can provide a basis for comparison of the relative adhesion of biomimetic CaP films on PE substrata.

UV irradiated samples show an overall decline in critical normal load values with increasing treatment time. However, the critical load is slightly higher than that for the control sample when the PE is treated for shorter times. Samples that had undergone glow-discharge processing with a forward power of 50 W showed a dramatic increase in critical normal load values when compared with the rest of the samples. The greatest critical normal load was achieved with a glow discharge treatment time of 30 seconds. Samples treated for 60 and 300 seconds showed a decrease in the critical normal load values, which still exceeded the values attained by the control sample and samples treated by UV irradiation.

Reductions in adhesion on samples that have undergone prolonged exposure to UV irradiation and/or glow discharge processing have been observed in previous work.^{11,19} Kokubo et al. hypothesized that extensive exposure of polymers to oxidizing environments actually compromises the structural integrity of the polymer surface.^{11-12,19} Degradation of the surface structure would invariably have a negative impact on the coatings adhered to it.

An alternative hypothesis to the structural degradation of the polymer is that the oxidation pretreatments actually remove the additives that act as nucleation sites inherent in the polymer. Once the removal of the surfactant reaches equilibrium, nucleation-enhancing polar groups begin to form in crystalline domains of the polymer. This hypothesis is further supported by the fact that glow discharge treatments increase the distribution of crystalline regions at the polymer surface since the amorphous phase is preferentially etched by the process.

The result that glow discharge processing enhances the substrate-coating adhesion characteristics of biomimetic CaP films on polyethylene substrates is in accordance with the integral work performed by Kokubo et al. They reported enhanced adhesion characteristics on a number of polymeric substrates after glow discharge processing due to the increased density of polar groups at the sample surface.^{11-12,19} Polar groups (carboxyls, carbonyls, hydroxyls, esters, etc.) produced by the glow discharge process act as strong nucleation sites for CaP formation. It follows that the increase in nucleation sites should improve the adhesion of the CaP films. The severe increase in the electron donor parameter (γ⁻) may be the underlying mechanism by which adhesion is improved by the production of these polar functional groups.

From these two studies the following conclusions can be formulated and used in the formation of an implant coating process for the delivery of biologically active substances, i.e. growth factors, bone morphogenic proteins (BMP) stem cells (osteoprogenitor cells, progenitor cells, bone marrow stromal cells, etc.)

### Conclusions

A. Self-assembled monolayers (SAMs) of alkanethiols served as model organic substrates in the biomimetic deposition of CaP coatings from supersaturated calcium phosphate solutions. It was found that homogeneous COOH-terminated SAMs and heterogeneous OH+CO OH SAMs produced CaP coatings that covered 100% of the substrate surface area within three days of chemical immersion. SAMs containing a predominantly methyl or hydroxyl character failed to elicit continuous CaP films covering all of the surface area. This trend of surface area coverage was observed regardless of the immersion method employed, which suggests that the deposition process is strongly dependent on surface functionality.
B. Both deposition methodology and substrate surface functionality had also influenced the chemical composition of the formed calcium phosphate coatings. Calcium to phosphorus ratios of CaP films varied from about 1.3 to 2.7 for coatings produced on SAMs of differing functionalities. OH-terminated SAMs induced formation of deposits with enriched calcium content whereas coatings with stoichiometry close to that for hydroxyapatite were preferentially formed on SAMs having COOH groups.
C. Oxidation pretreatments had little effect on the topography of the PE samples, but they did affect the underlying surface structure and chemistry. Contact angle analysis showed a significant shift in the hydrophobic character of the polymer to a more hydrophilic character upon substrate pretreatment. Among the substrate pretreatments, glow discharge processing lead to the greatest increase in hydrophilicity. Subsequent surface energy calculations based on the contact angle analysis revealed that glow discharge treated samples had the highest electron donor parameter (γ⁻) when compared to untreated PE samples and PE samples that had undergone UV irradiation.
D. CaP coatings deposited on the PE substrates were very porous and consisted of elongated clusters of spherical particles. 100% of the free surface area of nearly all of the substrates was covered with a CaP film after a three day immersion in a supersaturated calcium phosphate solution. Even the control substrate that had not undergone an oxidation pretreatment had 100% substrate coverage; a fact that suggests that adding lubricants or other additives of polar functionality can be sufficient for promoting adhesion sites for the nucleation and/or deposition of CaP.
E. This study suggests that orthopedic components made from polymers can be coated by a bioactive CaP coating through a simple biomimetic process. Adhesion of the coating to the polyethylene substrate can be enhanced by using glow-discharge processing of the polymer prior to immersion in a supersaturated calcium phosphate solution. It was determined that adhesion was most improved when PE substrates were subjected to 50 W glow discharge treatments. The glow discharge treated PE samples had the highest electron donor parameter of surface energy characteristic. Enhancing the electron donor parameter of the material seems to lead to improved substrate-coating adhesion.

### Processing Conditions

As the conclusions of the previous studies clearly suggest the ability to utilize substrate materials in their raw (as machined, as received) state and not requiring treatments to attain a specific level of contact surface roughness (as is clearly taught in prior art coating methods) has resulted in drastic reduction of production time, labor costs and material costs in the delivery system of the invention. However, this does not suggest that a good cleaning regime is not implemented. Failure to implement the specified cleaning regime in previous research led to the inability to deposit CaP films on metallic substrates. The presence of dust, organic contaminants from the laboratory environment may impart an unfavorable charge to the substrates thereby negating deposition of CaP on the substrates.

Dust particles and other organic contaminants may favor heterogeneous nucleation due to the reduction in free energy associated with the binding of CaP articles to the contaminants, rather than with the substrate itself. Accordingly, the process of the invention proposes the following substrate cleaning regime:

Substrate Cleaning Regime for Metals:
a. Ultrasonic cleaning in 70% ethanol for approximately 5 minutes.
b. Ultrasonic cleaning in diluted surfactant solution for approximately 5 minutes.
c. Ultrasonic cleaning in deionized water for approximately 5 minutes.
d. Rinse with acetone for approximately 30 seconds.
e. Air dry.

Substrate Cleaning Regime for Polymers:
a. Ultrasonic cleaning in 70% ethanol for approximately 5 minutes.
b. Ultrasonic cleaning in diluted surfactant solution for approximately 5 minutes.
c. Ultrasonic cleaning in deionized water for approximately 5 minutes.
d. Air dry for 10 minutes, or dry in oven at 50-100° C. for 5 minutes.

The cleaning regime is followed by a substrate pretreatment, which also differs for metals than for polymers. For metallic substrates, the invention proposes the use of a precalcification step (immersion in boiling CaOH₂) to aid in the formation of a calcium-titanate film, which acts to further complex with calcium ions when the substrate is subsequently immersed in a supersaturated calcium phosphate solution (SCPS). It is thought that the Ca²⁺ ions from the precalcification step will actually penetrate into the oxide film present on the metal substrate prior to treatment. These Ca²⁺ ions attract calcium phosphate clusters and may actually further supersaturate the SCPS in proximity to the substrate, thereby enhancing deposition of the CaP films.

Polymer substrates pretreatment according to the invention, proposes the use of glow-discharge processing. The use of glow-discharge (a.k.a. corona discharge, or plasma treating) processing has been used in several other industries to enhance the wettability of polymers in applications such as the improvement of ink printability and adhesion. In the application of the invention, it was found that glow discharge processing actually enhances a specific surface energy component, known as the electron donor parameter. The electron donor parameter is a quantification of the number of sites on a surface that will actively share electrons with another material. To the knowledge of the inventors, the electron donor parameter has not been addressed in any body of literature to date regarding the deposition of any type of coating on any substrate. This knowledge was gained only by experimentation (empirical evidence). Scission reactions occurring during glow-discharge processing are known to produce surface polar groups (carbonyl, carboxyl, esters, etc.), which impart specific effects to the surface energy and surface energy components of a particular material. Previous to our research, the effect of glow discharge processing on the deposition and adhesion of CaP films to polymeric substrates was investigated (Kokubo et al.). However, there was a failure to elucidate the exact mechanism by which glow discharge processing enhances deposition and adhesion of CaP films.

The above substrate pretreatments are summarized as follows:

Metallic Substrate Pretreatment:
a. After cleaning, immerse sample in a vessel containing a boiling, saturated calcium hydroxide solution making sure that the sample does not contact the vessel itself.
b. Keep sample immersed for approximately 10-30 minutes in the solution, preferably 20 minutes.
c. Remove from solution, rinse with deionized water and air dry, or dry in an oven at 100° C. for 10 minutes.

Polymeric Substrate Pretreatment:
a. After cleaning, subject the sample to glow discharge processing with a forward power of 10-300 Watts, preferably 50 W, in an O₂ saturated environment.
b. Duration of the treatment should be between 10-300 seconds.

Most prior art publications concerning the study of deposition of CaP films on various substances centers around the use of simulated body fluids (solution to mimic the composition of human blood plasma). Simulated body fluids (SBF) are principally used to study how bone growth will occur in vivo, by doing in vitro experiments. Since the objective of the invention is simply to prove a CaP coated substrate to use as a delivery vehicle, rather than understand how bone growth will occur, the invention proposes the use of a supersaturated calcium phosphate solution (SCPS) that contains only calcium and phosphate ions. It is felt that by reducing the number of different ionic species in solution, the industrial viability of the process is enhanced.

The composition of the SCPS is most important. In particular, the ratio of calcium to phosphorus in the SCPS plays a major role in the final composition of the coating. A ratio of 1.67 calcium to phosphorus is preferred to achieve the final composition of the coating. This ratio is equal to that of so-called stoichiometric hydroxyapatite. As set forth below, the SCPS is composed of 5.6 mM CaCl₂.2H₂O and 3.34 mM NaH₂PO₄.H₂O. Changing the calcium to phosphorus ratio of the solution may lead to changes in supersaturation of one ion with respect to another and change the final composition of the coating. Changes in this ratio may also affect the rate of speed of the deposition process, thereby leading to undesired changes in coating coverage, morphology and composition.

It is preferred that the SCPS is buffered to a pH of 7.40±0.05 for metallic and polymeric substrates. Theoretical work has shown that the thermodynamics and kinetics of CaP precipitation from a supersaturated solution are largely pH dependent. Slight changes in pH may yield the precipitation of specific phases of CaP that have different compositions, morphologies, crystal structures and solubility characteristics. Tris(hydroxymethyl)aminomethane is used in place of other types of buffers due to its ability to elevate and maintain the solution pH to specific levels with only small amounts of buffer.

The specific preparation of SCPS according to the invention is as follows:

Preparation of the Supersaturated Calcium Phosphate Solution (SCPS):
a. Prepare a CaCl₂ solution by dissolving reagent grade CaCl₂.2H₂O in deionized water.
b. Prepare a NaH₂PO₄ solution by dissolving reagent grade NaH₂PO₄.H₂O in deionized water.
c. Prepare the aqueous tris buffer by dissolving reagent grade tris(hydroxymethyl)aminomethane in deionized water.
d. Mix the CaCl₂ and NaH₂PO₄ in a 1:1 volumetric ratio under constant stirring to give an in-solution Ca:P ratio between 1.3 and 2.0, preferably 1.67.
e. Add the pH buffer (tris(hydroxymethyl)aminomethane) under constant magnetic stirring until a pH of 5.8-7.45, preferably 7.40±0.05 is attained.

f. Allow SCPS to reach 25° C.

Change in solution temperature effects the supersaturation of the solution, thereby influencing the rate of deposition of CaP films on the substrate. The use of a solution temperature of 25° C. was found to be ideal for the specific concentration of reagents used. Most prior art research have proposed the use of a physiological temperature of 37° C. during deposition experiments. Use of this elevated temperature with the above described invention procedure of the SCPS would reduce the supersaturation and retard the deposition process. Elevated temperatures also necessitate the expenditure of energy, which increases the processing costs associated with this biomimetic technique.

During early attempts to develop the process of the invention, substrates were simply laid flat on the bottom of a glass beaker filed with SCPS. Upon removal from the SCPS beaker, it was found that deposition preferentially occurred on the surface in contact with the glass beaker. Interactions between the solution, substrate and silicate ions in the glass skewed the results of experimentations. The proximity of the substrate to a flat surface may also predispose the system to intense local changes in solution chemistry (pH, ion concentration, etc.). These earlier mishaps resulted in the implementation of the use of a vertical immersion technique, as set forth below in detail, in which no part of the substrate is in contact with, nor is in any significant proximity to the sides of a container during immersion.

Immersion of Samples in SCPS
a. Suspend samples in SCPS making sure that the sample is not in contact with the walls of the vessel.
b. Cover the immersion vessel.
c. Leave samples in SCPS for 36-84 hours, preferably 72 hours, with solution refreshments taking place every 8-24 hours, preferably 24 hours.
d. Remove from solution, rinse with deionized water and dry at 50-100° C. for 10-15 minutes.

The final Ca:P ratio of the CaP films is approximately 1.34 which is significantly less than that of hydroxyapatite. From the result of the invention process, it can be confidently concluded that the invention process films are in the form of octacalcium phosphate (OCP) with some amorphous phases present. Most bioactive coatings used and reported in the prior art are in the form of hydroxyapatite. Prior art indicates that OCP is a precursor to hydroxyapatite. If hydroxyapatite is the desired phase of calcium phosphate for a particular application, extra time in the SCPS without solution refreshments will facilitate a phase transformation from OCP to hydroxyapatite.

By itself, the CaP films deposited via this biomimetic process act as a scaffold for bone growth on orthopedic implants. Thus, the biomimetic coatings add the attribute of osteoconductivity to the implant. To maximize bone growth a system must also induce bone growth, or possess the attribute of osteoinductivity. The inventors have shown that osteoinductive and therapeutic agents can be incorporated within the calcium phosphate structure during the coatings process.

In order to combine the attributes of osteoconductivity and osteoinductivity to the overall process by the use of biological active substances (growth factors, bone morphogenic proteins (BMP), stem cells-osteoprogenitor cells, progenitor cells, bone marrow cells, etc) it is necessary to propose minor additional considerations to the above set forth inventive process. For example, deposition experiments have shown a strong bonding affinity between CaP particles and osteogenic proteins in solution. This can be explained by electrostatic interactions between the proteins, Ca²⁺ and PO₄⁻ ions in the aqueous environment. It is hypothesized that the system free energy is reduced by complexation between the proteins and CaP particles, as described in classical equation of heterogeneous nucleation.

Maintaining the biological activity of therapeutic agents during their incorporation within the CaP matrix is of primary importance. The therapeutic agents are denatured by extremely acidic, or moderately basic environments. It has been observed that the range of pH values used in the described process does not reduce the biological activity of the incorporated agents. In fact, recent research suggests that the application of a BMP+CaP coating to resorbable polymeric substances may actually help preserve the potency of the BMPs, since the breakdown products produce a slightly acidic environment. For the addition of BMPs to the process, the substrate cleaning regime and pretreatment procedure as set forth hereinabove remains the same. The preparation of the supersaturated calcium phosphate solution (SCPS) adds an additional step as follows:
a. Prepare a CaCl₂ solution by dissolving reagent grade CaCl₂.2H₂O in deionized water.
b. Prepare a NaH₂PO₄ solution by dissolving reagent grade NaH₂PO₄.H₂O in deionized water.
c. Prepare the aqueous tris buffer solution by dissolving reagent grade tris(hydroxymethyl)aminomethane in deionized water.
d. Mix the CaCl₂ and NaH₂PO₄ solutions in a 1:1 volumetric ratio under constant stirring to give an in-solution Ca:P ratio between 1.3 and 2.0 (preferably 1.67)
e. Add the pH buffer solution (tris(hydroxymethyl)aminomethane) under constant magnetic stirring until a pH of 5.8-7.45, preferably 7.40±0.05 is attained.
f. Add lyophilized or liquefied growth factors, antibiotics, or BMPs to said solution.
g. Allow SCPS to reach 25° C.

The immersion process set forth above remains the same. However, the invention proposes an additional step after the immersion process is completed. Release profiles of BMPs from CaP films are characteristically bimodal, with an initially high diffusion rate that tapers off to a more constant rate over time.^{8,10,12} The second phase of release would sustain cellular activity for an extended period of time, leading to a higher probability of successful bone fusion. Secondary release would also enhance the rate of bone growth and confine the BMPs to a more local region of application. To support this bimodal release profile the invention proposes, as a final step after the immersion process is completed and immediately before implantation, that the implant materials having the BMP+CaP coating thereon is dipped in BMPs just prior to implantation. Once implanted, the BMPs coated on the implant materials as a result of the dipping step will provide an initial burst of cellular activity at the proposed fusion site due to its water-soluble nature of the site. Due to its characteristic bimodal nature rate of the release profile, after the initial burst of cellular activity the BMP's will begin to defuse away from the CaP coating and continue cellular activity at the fusion site. This cellular activity will be more localized and sustained for a longer period of time than the prior art practice of using a BMP soaked collagen sponge. The CaP film will begin to dissolve allowing for the usage of the calcium phosphate ions in the mineralization process. The CaP film will also act as a scaffold for new bone formation at the proposed fusion site. Degradation of the interbody device will allow for greater load-sharing to take place between the device and the biological system, which will enhance fusion quality and rate.

It is envisioned that the implant with a CaP+BMP coating thereon is also intended to be used as a delivery vehicle for stem and/or progenitor cells. Differentiation and adhesion of progenitor cells is enhanced by the nano-scale topography and favorable surface chemistry of the BMP+CaP films. While the exact mechanism is not clearly understood, it is hypothesized that the combination and frequency of micro- and nano-scale roughness of the coating enhances the adhesion of the cells. Surface energy parameters, such as the electron acceptor parameter may also explain the increase in differentiation and adhesion. For this application the same procedure as set forth above is used. The only additional step is that after the implant with the BMP+CaP co-precipitated coating thereon has been dipped in a BMP solution the coated device is allowed to dry and after the material has dried, the cells are cultured on the surface of the implant. The culturing process is not specific. Nothing that occurs during the culturing process will detrimentally affect the coatings or implant materials.

While the present invention has been described in terms of a preferred embodiment, it is apparent that other forms could be adopted by one skilled in the art. For example the teachings of the present invention encompass any reasonable substitutions or equivalents of claim limitations. Those skilled in the art will appreciate that other applications, including those outside of the biomedical industry, are possible with this invention. Accordingly, the present invention is not limited to only the disclosed preferred embodiments and its equivalents. Accordingly, the scope of the present invention is to be limited only by the following claims.

### REFERENCES

1. Bren L, Drelich J, English L, Fogarty J, Istephanous N, Policoro R, Zsidi A. Surface Characteristics of Metallic Biomaterials and Their Effect on Interaction with Osteoblast Cells. Podium Presentation at Biological Materials Science and Engineering: Biological Materials/Bio-Medical Applications II Symposium of the 2005 TMS Annual Meeting San Francisco, Calif.
2. Yang B, Uchida M, Kim H M, Zhang X, Kokubo T. Preparation of Bioactive Titanium Metal Via Anodic Oxidation Treatment. Biomaterials. 25 (2004) 1003-1010.
3. Uchida M, Kim H M, Miyaji F, Kokubo T, Nakamura T. Apatite Formation on Zirconium Metal Treated with Aqueous NaOH. Biomaterials. 23 (2002) 313-317.
4. Nishiguchi S, Kato H, Neo M, Oka M, Kim H M, Kokubo T, Nakamura T. Alkali- and Heat-Treated Porous Titanium for Orthopedic Implants. Journal of Biomedical Materials Research. 54 (2) (2001) 198-208.
5. Nishio K, Neo M, Akiyama H, Nishiguchi S, Kim H M, Kokubo T, Nakamura T. The Effect of Alkali- and Heat-Treated Titanium and Apatite-Formed Titanium on Osteoblastic Differentiation of Bone Marrow Cells. Journal of Biomedical Materials Research. 52 (4) (2000) 652-661.
6. Nishiguchi S, Kato H, Fujita H, Kim H M, Miyaji F, Kokubo T, Nakamura T. Enhancement of Bone-Bonding Strengths of Titanium Alloy Implants by Alkali and Heat Treatments. Journal of Biomedical Materials Research. 48 (5) (1999) 689-96.
7. Dunne N J, Orr J F. Effect of Mixing Method on the Fatigue Properties of Palacos R® Acrylic Bone Cement. Journal of Materials Science: Materials in Medicine 2002; 13:17-22.
8. Feith R. Side-Effects of Acrylic Cement Implanted into Bone: a Histological, (Micro)Angiographic, Fluorescence-Microscopic and Autoradiographic Study in the Rabbit Femur. Acta Orthopaedica Scandinavica 1975; suppl.: 161.
9. De Wijn J R, Driessens F C, Slooff T J. Characterization of Bone Cements. Journal of Biomaterials Research 1975; 6:99-103.
10. Baker K C, Anderson M A, Oehlke S A, Astashkina A I, Haikio D C, Donahue S W, Drelich J W. 2005. Growth, Characterization and Biocompatibility of Bone-Like Apatite Layers Biomimetically Deposited on Metallic Substrata. Journal of Materials Science and Engineering Part C 2006; 26:1351-1360.
11. Tanahashi M, Yao T, Kokubo T, Minoda M, Miyamota T, Nakamura T, Yamamuro T. 1995. Apatite Coated on Polymers by Biomimetic Process: Improvement In Its Adhesion to Substrate by Glow-Discharge Treatment. Journal of Biomedical Materials Research. 1995 Volume 29, 349-357.
12. Tanahashi M, Yao T, Kokubo T, Minoda M, Miyamoto T, Nakamura T, Yamamuro T. Apatite Coated on Organic Polymers by Biomimetic Process: Improvement in its Adhesion to Substrate by NaOH Treatment. Journal of Biomaterials: Applied Biomaterials 1994; 5:339-347.
13. Liu Y, Hunziker E B, Van de Vaal C, de Groot K. 2003. Biomimetic Coatings vs. Collagen Sponges as a Carrier for BMP-2: a Comparison of the Osteogenic Responses Triggered In Vivo Using an Ectopic Rat Model. Bioceramics: Key Engineering Materials 2002; 16:245-256.
14. Barbanti S H, Santos A R, Zavaglia C A, Duek E A. 2004. Porous and Dense Poly(L-Lactic Acid) and Poly(D,L-Lactic Acid-Co-Glycolic Acid) Scaffolds: In Vitro Degradation in Culture Medium and Osteoblasts Culture. Journal of Materials Science: Materials in Medicine (2004) Volume 15, 1315-1321.
15. Kim H W, Knowles J C, Kim B E. 2005. Hydroxyapatite Porous Scaffold Engineered With Biological Polymer Hybrid Coating for Antibiotic Vancomycin Release. Journal of Materials Science: Materials in Medicine. (2005) Volume 16, 189-195.
16. Wen B B, de Wijn J R, van Blitterswijk A, de Groot K. Incorporation of Bovine Serum Albumin in Calcium Phosphate Coating on Titanium. Journal of Biomedical Materials, Research: Part B, Volume 46, (1999) No. 2, 245-252.
17. Rohanizade R, LeGeros R Z, Harsono M, Bendavid A. Adherent Apatite Coating on Titanium Substrate Using Chemical Deposition. Journal of Biomedical Materials Research: Part A. 72 (4) (2005) 428-438.
18. Liu Q, Ding J, Mante F K, Wunder S L, Baran G R. 2002. The Role of Surface Functional Groups in Calcium Phosphate Nucleation on Titanium Foil: A Self-Assembled Monolayer Technique. Biomaterials. Volume 23, 3103.
19. Liu G J, Miyaji F, Kokubo T, Takadama H, Nakamura T, Murakami A. 1998. Apatite-Organic Polymer Composites Prepared by a Biomimetic Process: Improvement of the Adhesion of the Apatite Layer to the Substrate by Ultraviolet Irradiation. Journal of Materials Science: Materials in Medicine. 1998 Volume 9, 285.
20. Tanahashi M and Matsuda T. 1997. Surface Functional Group Dependence on Apatite Formation on Self-Assembled Monolayers in a Simulated Body Fluid, Journal of Biomedical Materials Research Volume 34, 305.
21. Sato K, Kumagai Y, Tanaka J. 2000. Apatite Formation on Organic Monolayers in Simulated Body Environment. Journal of Biomedical Materials Research Volume 50, 16.
22. Drelich J, Miller J D, and Good R J. 1996. The Effect of Drop (Bubble) Size on Advancing and Receding Contact Angles for Heterogeneous and Rough Solid Surfaces as Observed with Sessile-Drop and Captive-Bubble Techniques. Journal of Colloid Interface Science 1996 Volume 179, 37.
23. Tormoen G W, Drelich J, and Beach E R. 2004. Analysis of Atomic Force Microscope Pull-off Forces for Gold Surfaces Portraying Nanoscale Roughness and Specific Chemical Functionality. Journal of Adhesion Science and Technology Volume 18, Number 1, 1.
24. Bain C D, Troughton E B, Tao Y-T, Evall J, Whitesides G M, and Nuzzo R G. 1989. Formation of Monolayer Films by the Spontaneous Assembly of Organic Thiols from Solution onto Gold. J. Am. Chem. Soc. 111, 321.
25. Folkers J P, Laibinis P E, Whitesides G M. 1992. Self-Assembled Monolayers of Alkanethiols on Gold: Comparisons of Monolayers Containing Mixtures of Short and Long-Chain Constituents with CH₃ and CH₂OH Terminal Groups. Langmuir Volume 8, 1330.
26. Tarasevich B J, Chusuei C C, Allara D L. 2003. Nucleation and Growth of Calcium Phosphate from Physiological Solutions onto Self-Assembled Templates by a Solution-Formed Nucleus Mechanism. Journal of Physical Chemistry: Part B. Volume 107, 10367.
27. Vatanatham M, Kimura S. 2000. Hydroxyapatite Coating on Thermally Oxidized Titanium Substrates. Journal of American Ceramic Society Volume 83, 2135.
28. Habibovic P, Barrere F. van Blitterswijk C, de Groot K, Layrolle P 2002. Biomimetic Hydroxyapatite Coating on Metal Implants. Journal of American Ceramic Society 2002 Volume 85, 517.
29. Drummond J L, Steinberg A D, Krauss A R. 1997. X-ray Photoemission and Energy Dispersive Spectroscopy of Hydroxyapatite-Coated Titanium. Journal of American Ceramic Society 1997 Volume 80, 1868.
30. Giavaresi G, Fini M, Cigada A, Chiesa R, Rondelli G, Rimondini L, Torricelli P, Aldini N N, Giardino R. 2003. Mechanical and Histomorphometric Evaluations of Titanium Implants with Different Surface Treatments Inserted in Sheep Cortical Bone. Biomaterials 2003 Volume 24, 1583.
31. Fini M, Giavaresi G, Greggi T, Martini L, Aldini N N, Parisini P, Giardini R. 2003. Biological Assessment of the Bone-Screw Interface After Insertion of Uncoated and Hydroxapatite-Coated Pedicular Screws in the Osteopenic Sheep. Journal of Biomedical Materials Research: Part A Volume 66, 176.
32. Ratner B D, Hoffman A S, Schoen F J, Lemons J E. Biomaterials Science. New York: Academic Press, 1996.
33. Conditt M A, Ismaily S K, Alexander J W, Noble P C. Backside Wear of Modular Ultra-High Molecular Weight. Polyethylene Tibial Inserts. J Bone and Joint Surg—American 2004; 86-A: 1031.
34. Conditt M A, Thompson M T, Usrey M M, Ismaily S K, Noble P C. Backside Wear of Polyethylene Tibial Inserts: Mechanism and Magnitude of Material Loss. J Bone and Joint Surg—American 2005; 87:326.
35. Zhu Y H, Chiu K Y, Tang W M. Polyethylene Wear and Osteolysis in Total Hip Arthroplasty. J Orthopaedic Surg 2001; 9:91.
36. Cheng K, Pruitt L, Zaloudek C, Ries M D. Osteolysis Caused by Tibial Component in Total Knee Arthroplasty. Clinical Orthopedics and Related Research 2006; 443:333.
37. Rohanizadeh R, Al-Sadeq M, Legeros R. Titanium Oxide Layers Obtained by Different Methods: Effect on Apatite Deposition. Key Engineering Materials 2003; 240:449.
38. Bren L, Drelich J, English L, Fogarty J, Istephanous N, Policoro R, Zsidi A. Effect of Surface Characteristics of Metallic Biomaterials on Interaction with Osteoblast Cells. Proceedings of the 7th World Biomaterials Congress, 17-21 May 2004, Sydney, Australia, p. 1121.
39. Kokubo T, Miyaji F, Kim H M. Spontaneous Formation of Bone-Like Apatite Layer on Chemically Treated Titanium Metals. Journal of American Ceramic Society. 1996; 79:1127.
40. Liang B, Fujibayashi S, Tamura J, Neo M, Kim H M, Uchida M, Kokubo T, Nakamura T. Histological and Mechanical Investigation of the Bone-Bonding Ability of Anodically Oxidized Titanium in Rabbits. Key Engineering Materials 2003; 240:923.
41. Kokubo T, Kim H M, Nishiguchi S, Nkamura T. Formation of Bioactive Functionally Graded Structure on Ti-6Al-4V Alloy by Chemical Surface Treatment. Key Engineering Materials 2000; 192:3.
42. Engin N O, Tas A C. Preparation of Porous Ca₁₀(PO₄)₆(OH)₂ and Ca₃(PO₄)₂ Bioceramics. Journal of the American Ceramic Society 2000; 83:1581.
43. Kannan S, Balamurugan A, Rajeswari S. Development of Calcium Phosphate Coatings on Type 316L S and Their In Vitro Response. Trends Biomater. Artif. Organs 2002; 16:8.
44. Mavis B, Tas A C. Dip Coating of Calcium Hydroxyapatite on Ti6Al4V Substrates. Journal of the American Ceramic Society 2001; 84:2135.
45. C. J. van Oss, Interfacial Forces in Aqueous Media, Marcel Dekker, Inc. New York 1994.
46. Goldman M, Gronsky R, Pruitt L. The Influence of Sterilization Technique and Ageing on the Structure and Morphology of Medical Grade Ultra-High Molecular Weight. Polyethylene. Journal of Materials Science: Materials Science: Materials in Medicine 1998; 4:207.
47. Lusietto Y, Wesslen B, Maurer F, Lidgren L. The Effect of Irradiation, Annealing Temperature, and Artificial Aging on the Oxidation, Mechanical Properties, and Fracture Mechanisms of UHMWPE. J Biomedical Materials Research 2003; 3:908.
48. Holmes-Farley S R, Bain C D, Whitesides G M. Wetting of Functionalized Polyethylene Film Having Ionizable Organic Acids and Bases at the Polymer-Water Interface: Relations Between Functional Group Polarity, Extent of Ionization and Contact Angle with Water. Langmuir 1998; 4:921.
49. Williams J G. Michigan Technological University. May 2005. Private Communication.
50. Saiz E, Goldman M, Tomsia A P. “FTIR Analysis of Apatite Formation on Bioactive Glass Coatings on Ti Alloys.” http://infrared.als.lbl.gov/pubs/GoldmanCompedium99.pdf

What is claimed is:

1. A method of making an implant device, said method comprising the steps of:
provide a substrate having an outer surface;
clean said outer surface of said substrate;
treat said outer surface to enhance the deposition of calcium ions;
prepare a supersaturated calcium phosphate solution; and
precipitate a calcium phosphate layer from said supersaturated calcium phosphate solution onto said outer surface of said substrate;
whereby said calcium phosphate layer precipitated onto said outer surface of said substrate acts as a scaffold for tissue growth.

2. The method of making an implant device as claimed in claim 1 wherein said step of precipitating said calcium phosphate layer onto said outer surface of said substrate further comprises:
suspend said outer surface of said substrate in an upright vessel containing said supersaturated calcium phosphate solution for 36-84 hours;
refresh said supersaturated calcium phosphate solution approximately every 8-24 hours;
thereafter remove said outer surface of said substrate from said supersaturated calcium phosphate solution and rinse with deionized water; and
dry said outer surface of said substrate at 50-100° C. for 10-15 minutes.

3. The method of making an implant device as claimed in claim 1 wherein said step of preparing further comprises the step of:
prepare a supersaturated calcium phosphate solution (SCPS) containing a therapeutic agent; and
wherein further said precipitating step comprises the step of co-precipitating a calcium phosphate layer from said supersaturated calcium phosphate and therapeutic agent solution onto said treated outer surface of said substrate.

4. The method of making an implant device as claimed in claim 3 wherein said step of co-precipitating said calcium phosphate layer onto said outer surface of said substrate further comprises:
suspend said outer surface of said substrate in an upright vessel containing said supersaturated calcium phosphate and therapeutic agent solution for 36-84 hours;
refresh said supersaturated calcium phosphate and therapeutic agent solution approximately every 8-24 hours;
remove said outer surface of said substrate from said supersaturated calcium phosphate and therapeutic agent solution and rinse with deionized water; and
dry said outer surface of said substrate at 50-100° C. for 10-15 minutes.

5. The method of making an implant device as claimed in claim 4 further comprising the step of immediately before implantation, dipping said coated outer surface of said substrate in a separate concentrated solution of said therapeutic agent so as to load a separate coating of said therapeutic agent on said coated surface of said substrate of said implant device whereby once implanted, said separate coating of said therapeutic agent will provide an initial burst of cellular activity at the proposed fusion site due to the water-soluble nature of said fusion site, and thereafter said supersaturated calcium phosphate solution with said therapeutic agent co-precipitated therewith will begin to diffuse away and continue cellular activity at said fusion site.

6. The method as claimed in claim 2 wherein said step of preparing a supersaturated calcium phosphate solution (SCPS) further comprises:
prepare a CaCl₂ solution by dissolving reagent grade CaCl₂.2H₂O in deionized water;
prepare a NaH₂PO₄ solution by dissolving reagent grade NaH₂PO₄.H₂O in deionized water;
prepare an aqueous tris-buffer by dissolving reagent grade tris(hydroxymethyl)-aminomethane in deionized water;
mix said CaCl₂ solution and said NaH₂PO₄ solution in a 1:1 volumetric ratio under constant stirring to give an in-solution Ca:P ratio between 1.3 and 2.0;
add the pH buffer under constant magnetic stirring until a pH of 5.8-7.45 is attained;
add lyophilized or liquefied therapeutic agent to said SCPS; and
allow said SCPS to reach 25° C. temperature.

7. The method of making an implant device as claimed in claim 3 wherein said step of preparing a supersaturated calcium phosphate solution (SCPS) further comprises:
prepare a CaCl₂ solution by dissolving reagent grade CaCl₂.2H₂O in deionized water;
prepare a NaH₂PO₄ solution by dissolving reagent grade NaH₂PO₄.H₂O in deionized water;
prepare an aqueous tris-buffer by dissolving reagent grade tris(hydroxymethyl)-aminomethane in deionized water;
mix said CaCl₂ solution and said NaH₂PO₄ solution in a 1:1 volumetric ratio under constant stirring to give an in-solution Ca:P ratio between 1.3 and 2.0;
add the pH buffer under constant magnetic stirring until a pH of 5.8-7.45 is attained;
add lyophilized or liquefied therapeutic agent to said SCPS; and
allow said SCPS to reach 25° C. temperature.

8. The method of making an implant device as claimed in claim 1 wherein said substrate is metal.

9. The method of making an implant as claimed in claim 8 wherein said step of cleaning further comprises:
ultrasonically cleaning said outer surface of said substrate in 70% ethanol for approximately five minutes;
ultrasonically cleaning said outer surface of said substrate in diluted surfactant solution for approximately five minutes;
ultrasonically cleaning said outer surface of said substrate in deionized water for approximately five minutes;
rinse said outer surface of said substrate with acetone for approximately 30 seconds; and
air dry said outer surface of said substrate.

10. The method of making an implant device as claimed in claim 1 wherein said substrate is a polymer.

11. The method of making an implant device as claimed in claim 10 wherein said step of cleaning further comprises:
ultrasonically cleaning said outer surface of said substrate in 70% ethanol for approximately five minutes;
ultrasonically cleaning said outer surface of said substrate in diluted surfactant solution for approximately five minutes;
ultrasonically cleaning said outer surface of said substrate in deionized water for approximately five minutes; and
air dry for ten minutes.

12. The method of making an implant device as claimed in claim 9 wherein said step of treating further comprises:
immersing said outer surface of said substrate in a vessel containing a boiling saturated calcium hydroxide solution for a duration of approximately 10-30 minutes;
remove said outer surface of said substrate from said boiling saturated calcium hydroxide solution and rinse with deionized water; and
air dry said outer surface of said substrate.

13. The method of making an implant device as claimed in claim 11 wherein said step of treating further comprises after said step of cleaning, subject said outer surface of said substrate to glow discharge processing with a forward power of 10-300 watts in an O₂ saturated environment for a duration of approximately 10-300 seconds.

14. The method of making an implant device as claimed in claim 1 further comprising the steps of culturing cells on said coated outer surface of said substrate.

15. The method of making an implant device as claimed in claim 3 further comprising the step of culturing cells on said coated outer surface of said substrate.

16. The method of making an implant device as claimed in claim 4 further comprising the step of culturing cells on said coated outer surface of said substrate.

17. The method of making an implant device as claimed in claim 12 further comprising the step of culturing cells on said coated outer surface of said substrate after said coated outer surface of said substrate has air dried.

18. The method of making an implant device as claimed in claim 13 further comprising the step of culturing cells on said coated outer surface of said substrate after said coated surface has air dried.

19. The method of making an implant device as claimed in claim 3 wherein said therapeutic agent is selected from a group consisting of growth factors, lipids, (hydro)polysaccharides, hormones, cytostatic agents, antibiotics, proteins, and mixtures thereof.

20. A method of creating a biomimetic coating on the surface of a polymeric implant device, said method comprising the steps of:
clean said surface of said polymeric implant device pretreat said polymeric implant device by subjecting said polymeric implant device to an oxidation pretreatment resulting in the formation of carboxyl (COOH) functional groups on said surface of said polymeric implant device;
immerse said polymeric implant device in a vessel containing supersaturated calcium phosphate solution (SCPS) combined with a biologically active substance so as to co-precipitate from said SCPS said biologically active substance plus a CaP coating on said surface of said polymeric implant device.

21. A method of making a biomimetic coating on the surface of an implant device, said method comprising the steps of:
ultrasonically cleaning said surface of said implant device;
pretreating said implant device by immersing said implant device in a vessel containing saturated calcium hydroxide solution; and
following said pretreating step, immerse said implant device in a vessel containing supersaturated calcium phosphate solution (SCPS) allowing a coating to precipitate from said SCPS onto said implant device

22. An implant device comprising:
a substrate base material having an outer surface;
a precalcification layer chemically attached to said outer surface of said substrate base material for said implant device such that said pre-calcification layer enhances the deposition of a calcium phosphate layer; and
a calcium phosphate layer precipitated onto said precalcification layer on said outer surface of said substrate base material, said calcium phosphate layer being precipitated from a supersaturated calcium phosphate solution containing only calcium and phosphate ions, whereby said calcium phosphate layer is in the form of an octacalcium phosphate film.

23. A polymeric implant device comprising:
a substrate base material having an outer surface;
means for subjecting said outer surface to an oxidation pretreatment resulting in the formation of carboxyl (COOH) functional groups at said outer surface of said substrate base material whereby the electron donor parameter of said outer surface is enhanced; and
a calcium phosphate layer precipitated onto said oxidation pretreated outer surface of said substrate base material of said polymeric implant device, said calcium phosphate layer being precipitated from a supersaturated calcium phosphate solution containing only calcium and phosphate ions so as to provide said calcium phosphate precipitated layer in the form of an octacalcium phosphate film.

24. An implant device adapted to be applied to human or animal tissue, said implant device comprising:
a substrate;
means for cleaning said substrate;
means for pretreating said substrate to enhance the deposition of calcium ions; and
a calcium phosphate layer precipitated from a supersaturated calcium phosphate solution onto said substrate;
whereby said calcium phosphate layer precipitated onto said substrate acts as a scaffold for tissue growth on said implant device.

25. The method of creating a biomimetic coating on the surface of a polymeric implant device, said method comprising the step of:
subjecting said surface of said polymeric implant device to glow discharge processing to enhance the electron donor parameter of said polymeric implant device whereby scission reactions occurring during said glow discharge processing produce surface polar groups (carbonyl, carboxyl, esters, etc.) which enhances the number of sites on said surface of said polymeric implant device that will actively share said electrons with another material; and
precipitating a calcium phosphate layer from a supersaturated calcium phosphate solution onto said surface of said polymeric implant device.

26. A method of making a metal implant device, said method comprising the steps of:
providing a substrate having an outer surface;
cleaning said outer surface in
70% ethanol for approximately five minutes; thereafter in
diluted surfactant solution for approximately five minutes; and thereafter in
deionized water for approximately five minutes;
rinsing said substrate with acetone for approximately 30 seconds;
air dry said substrate;
immerse said outer surface of said substrate in a vessel containing a boiling saturated calcium hydroxide solution avoiding said outer surface from contacting said vessel;
said immersing step duration continuing for approximately 10-30 minutes;
remove said metal implant device from said calcium hydroxide solution and rinse with deionized water;
air dry said outer surface of said substrate;
prepare a supersaturated calcium phosphate solution (SCPS); and
precipitate a calcium phosphate layer from said supersaturated calcium phosphate solution onto said outer surface of said substrate by immersing said outer surface of said substrate in a vessel containing said SCPS;
whereby said calcium phosphate layer precipitated onto said outer surface of said substrate acts as a scaffold for tissue growth.

27. A method of making a polymeric implant device, said method comprising the steps of:
providing a substrate having an outer surface;
cleaning said outer surface of said substrate in
70% ethanol for approximately five minutes; thereafter in
diluted surfactant solution for approximately five minutes; and thereafter in
deionized water for approximately five minutes;
air drying said outer surface of said substrate;
treat said outer surface of said substrate to glow discharge processing with a forward power of approximately 10-300 watts in an O₂ saturated environment for a duration of approximately 10-300 seconds;
prepare a supersaturated calcium phosphate solution (SCPS);
precipitate a calcium phosphate layer from said supersaturated calcium phosphate solution onto said treated outer surface of said substrate by immersing said outer surface of said substrate in a vessel containing said SCPS;
whereby said calcium phosphate layer precipitated onto said outer surface of said substrate acts as a scaffold for tissue growth.
